(19) United States
(12) Patent Application Publication
(10) Pub. No.: US 2005/0130902 A1
(43) Pub. Date: Jun. 16, 2005

(54) Peptide compounds for counteracting reactive oxygen species and free radicals

(76) Inventor: Victor E. Shashoua, Brookline, MA (US)

Correspondence Address: Brown Raysman Millstein Felder & Steiner, 900 Third Avenue, New York, NY, 10022, US

(21) Appl. No.: 10/477,683

(22) PCT Filed: May 17, 2002
(86) PCT No.: PCT/US02/15790

Related U.S. Application Data
(60) Provisional application No. 60/291,673, filed on May 17, 2001.

(51) Int. Cl. A61K 38/08; A61K 38/06

(52) U.S. Cl. 514/17; 514/18

(57) ABSTRACT

The invention provides compositions comprising isolated peptide compounds, which upregulate expression of a gene encoding an antioxidative anzyme, such as superoxide dismutase or catalase, to counteract harmful oxidative effects of reactive oxygen species and other free radicals. The composition may be used to treat or prevent diseases and conditions characterized by undesirable elevation of reactive oxygen species and other free radicals.

### FIELD OF THE INVENTION

The present invention is in the field of antioxidative compounds, in particular, compounds for use in therapeutic and prophylactic treatments of diseases and conditions characterized by undesirable levels of reactive oxygen species and free radicals.

### BACKGROUND OF THE INVENTION

Biological organisms generate harmful reactive oxygen species (ROS) and various free radicals in their cells and tissues in the course of normal metabolic activities (Halliwell, B. and Gutteridge, J. M. C., eds., In Free Radicals in Biology and Medicine, (Oxford: Clarendon Press, 1989)). The most reactive and, therefore, toxic ROS and free radicals include the superoxide anion (O₂.⁻.), singlet oxygen, hydrogen peroxide (H₂O₂), lipid peroxides, peroxinitrite, and hydroxyl radicals. Even a relatively small elevation in ROS or free radical levels in a cell can be damaging. Likewise, a release or increase of ROS or free radicals in extracellular fluid can jeopardize the surrounding tissue and result in tissue destruction and necrosis. Indeed, hydrogen peroxide, which is somewhat less reactive than the superoxide anion, is a well known, broad spectrum, antiseptic compound. In eukaryotes, a major source of superoxide anion is the electron transport system during respiration in the mitochondria. The majority of the superoxide anion is generated at the two main sites of accumulation of reducing equivalents, i.e., the ubiquinone-mediated and the NADH dehydrogenase-mediated steps in the electron transport mechanism. Hydrogen peroxide is generated metabolically in the endoplasmic reticulum, in metal-catalyzed oxidations in peroxisomes, in oxidative phosphorylation in mitochondria, and in the cytosolic oxidation of xanthine (see, e.g., Somani et al., “Response of Antioxidant System to Physical and Chemical Stress,” In Oxidants, Antioxidants, and Free Radicals, chapter 6, pp. 125-141, Baskin, S. I. and H. Salem, eds. (Taylor & Francis, Washington, D.C., 1997)).

In normal and healthy individuals, several naturally occurring antioxidant defense systems detoxify the various ROS or free radicals and, thereby, preserve normal cell and tissue integrity and function. These systems of detoxification involve the stepwise conversion of ROS or free radicals to less toxic species by the concerted activities of certain antioxidative enzymes. These antioxidative enzymes are members of a larger class of molecules known as “oxygen radical scavengers” or “lazaroids” that have an ability to scavenge and detoxify ROS and free radicals. Vitamins A, C, E, and related antioxidant compounds, such as β-carotene and retinoids, are also members of this larger class. In healthy individuals, sufficient levels of antioxidative enzymes and other lazaroids are present both intracellularly and extracellularly to efficiently scavenge sufficient amounts of ROS and free radicals to avoid significant oxidative damage to cells and tissues.

Superoxide dismutase (SOD), catalase (CAT) and glutathione peroxidase (GSH-Px) are among the most important and studied of the antioxidative enzymes. These enzymes function in concert to detoxify ROS and free radicals. SOD is present in virtually all oxygen-respiring organisms where its major function is the dismutation (breakdown) of superoxide anion to hydrogen peroxide. Hydrogen peroxide, itself, is a highly reactive and oxidative molecule, which must be further reduced to avoid damage to cells and tissues. In the presence of the appropriate electron acceptors (hydrogen donors), CAT catalyzes the further reduction of hydrogen peroxide to water. In the presence of reduced glutathione (GSH), GSH-Px also mediates reduction of hydrogen peroxide to water by a separate pathway.

Each of the antioxidative enzymes described above can be further subdivided into classes. There are three distinct classes of SOD based on metal ion content: copper-zinc (Cu—Zn), manganese (Mn), and iron (Fe). In mammals, only the Cu—Zn and Mn SOD classes are present. Mammalian tissues contain a cytosolic Cu—Zn SOD, a mitochondrial Mn SOD, and a Cu—Zn SOD referred to as EC-SOD, which is secreted into the extracellular fluid. SOD is able to catalyze the dismutation of the highly toxic superoxide anion at a rate that is 10 million times faster than the spontaneous rate (see, Somani et al., p. 126). Although present in virtually all mammalian cells, the highest levels of SOD activity are found in several major organs of high metabolic activity, i.e., liver, kidney, heart, and lung. Expression of the gene encoding SOD has been correlated with tissue oxygenation; high oxygen tension elevates SOD biosynthesis in rats (Toyokuni, S., Pathol. Int., 49: 91-102 (1999)).

CAT is a soluble enzyme present in nearly all mammalian cells, although CAT levels can vary widely between tissues and intracellular locations. CAT is present predominately in the peroxisomes (microbodies) in liver and kidney cells and also in the microperoxisomes of other tissues.

There are two distinct classes of GSH-Px: selenium-dependent and selenium independent. Furthermore, GSH-Px species can be found in the cytosol, as a membrane-associated protein, and as a circulating plasma protein.

A recognition of the role of ROS and free radicals in a variety of important diseases and drug side effects has grown appreciably over recent years. Many studies have demonstrated that a large number of disease states and harmful side effects of therapeutic drugs are linked to a failure of the antioxidant defense system of an individual to keep up with the rate of generation of ROS and various free radicals (see, for example, Chan et al., Adv. Neurol., 71:271-279 (1996); DiGuiseppi, J. and Fridovich, I., Crit. Rev. Toxicol., 12:315-342 (1984)). For example, abnormally high ROS levels have been found under conditions of anoxia elicited by ischemia during a stroke or anoxia generated in heart muscle during myocardial infarction (see, for example, Walton, M. et al., Brain Res. Rev., 29:137-168 (1999); Pulsinelli, W. A. et al., Ann. Neurol., 11: 499-502 (1982); Lucchesi, B. R., Am. J. Cardiol., 65:141-231 (1990)). In addition, an elevation of ROS and free radicals has also been linked with reperfusion damage after renal transplants. Moreover, an increasing number of studies have shown a correlation between oxidative tissue damage and age-associated brain dysfunction, as evidenced by age-related loss of various cognitive and motor functions, and/or a progressive increase in oxidatively modified DNA and proteins during aging (see, e.g., Coyle et al., Science, 262: 689-695 (1993); Halliwell, J. Neurochem., 59: 1609-1623 (1992); Sohal et al., Free Radical Biol. Med., 10: 495-500 (1990); Sohal et al., Mech. Ageing Dev., 76: 215-224 (1994); Ames, Free Radical Res. Commun., 7: 121-128 (1989); and Stadtman, Science, 257: 1220-1224 (1992)). Accordingly, an elevation of ROS and free radicals has been linked with the progression and complications developed in many diseases, drug treatments, traumas, and degenerative conditions, including age-related oxidative stress induced damage, Tardive dyskinesia, Parkinson's disease, Huntington's disease, degenerative eye diseases, septic shock, head and spinal cord injuries, Alzheimer's disease, ulcerative colitis, human leukemia and other cancers, and diabetes (see, e.g., Ratafia, Pharmaceutical Executive, pp. 74-80 (April 1991)).

One approach to reducing the damaging effects of elevated levels of ROS and free radicals, including the effect of oxidative damage in the aging process, has been to increase the uptake of lazaroids to scavenge or reduce ROS and free radicals. As a result, the commercial market for antioxidative enzymes and other lazaroids is estimated to exceed $1 billion worldwide. Not surprisingly, research and development of various lazaroids as therapeutic agents has become a highly competitive field (see, Ratafia, Pharmaceutical Executive, pp. 74-80 (April 1991)). Interest in developing SOD, itself, as a therapeutic agent has been especially strong. This is due, in part, to SOD's status as a recognized anti-inflammatory agent and the belief that SOD might provide a means for penetrating the nonsteroidal, anti-inflammatory drug (NSAID) market as well (see, Ratafia, Pharmaceutical Executive, pp. 74-80 (April 1991)).

Despite many years of focused research effort, the use of SOD and other lazaroids has not provided a successful prophylactic or therapeutic tool for addressing the diseases, disorders and other conditions caused by or characterized by the generation of ROS and free radicals. Clearly, there remains a need for additional therapeutics and methods of treating diseases and conditions characterized by the destructive effect of elevated levels of ROS and free radicals.

### SUMMARY OF THE INVENTION

The invention described herein solves the problem of how to counteract the destructive oxidative effect of elevated levels of ROS and free radicals by providing compositions comprising peptide compounds that stimulate (i.e., upregulate) expression of genes encoding antioxidative enzymes, such as superoxide dismutase (SOD) and/or catalase (CAT), to reduce, eliminate, or prevent an undesirable elevation in the levels of ROS and free radicals in cells and tissues, and to restore age-related reduction of constitutive antioxidative enzymes. Furthermore, the peptide compounds of this invention may have antioxidative activity independent of their ability to stimulate expression of genes encoding antioxidative enzymes. The formulas and sequences of the peptide compounds described herein use the standard three-letter or one-letter abbreviations for amino acids known in the art.

In one embodiment, the invention provides a composition comprising an isolated peptide compound having the formula:
R₁ Asp Gly Xaa₃ Xaa₄ Xaa₅ R₂ (SEQ ID NO:1),
wherein R₁ is absent or is an amino terminal capping group; Xaa₃ is Glu or Leu; Xaa₄ is Ala or Glu; Xaa₅ is absent, Leu, or Ala; and R₂ is absent or is a carboxy terminal capping group of the peptide compound; and wherein the peptide compound upregulates expression of a gene encoding an antioxidative enzyme.

In a preferred embodiment, compositions of the invention comprise an isolated peptide compound of any of the following formulas:

| | |
|---|---|
| R₁ Asp Gly Glu Ala R₂, | (SEQ ID NO: 2) |
| R₁ Asp Gly Glu Ala Leu R₂, | (SEQ ID NO: 3) |
| R₁ Asp Gly Leu Glu Ala R₂, | (SEQ ID NO: 4) |

wherein R₁ is absent or is an amino terminal capping group of the peptide compound and R₂ is absent or is a carboxy terminal capping group of the peptide compound; and wherein the peptide compound upregulates expression of a gene encoding an antioxidative enzyme.

In another embodiment, a composition of the invention comprises an isolated peptide compound having any of the following amino acid sequences:

| | |
|---|---|
| Asp Gly Glu Ala, | (SEQ ID NO: 2) |
| Asp Gly Glu Ala Leu, | (SEQ ID NO: 3) |
| Asp Gly Leu Glu Ala, | (SEQ ID NO: 4) |

wherein the peptide compound upregulates expression of a gene encoding an antioxidative enzyme.

Peptide compounds of the invention may contain one or more additional amino acids linked at the amino terminal and/or carboxy terminal amino acids of a “core sequence” of amino acids of the peptide compounds, such as the amino acid sequences of SEQ ID NOS:2, 3, and 4, provided the peptide compound still upregulates expression of a gene encoding an antioxidative enzyme to increase antioxidative enzyme activity in a cell or tissue. In a preferred embodiment, a composition of the invention comprises an isolated peptide compound having the formula:
R₁ Xaa₁ Xaa₂ Asp Gly Xaa₅ Xaa₆ Xaa₇ Xaa₈ Xaa₉ Xaa₁₀ Xaa₁₁ R₂ (SEQ ID NO:5),
wherein R₁ is absent or is an amino terminal capping group; Xaa₁ is absent or any amino acid; Xaa₂ is absent or any amino acid; Xaa₅ is Glu or Leu; Xaa₆ is Ala or Glu; Xaa₇ is absent, Leu, or Ala; Xaa₈ is absent or is any amino acid; Xaa₉ is absent or is any amino acid; Xaa₁₀ is absent or is any amino acid; Xaa₁₁ is absent or is any amino acid; and R₂ is absent or is a carboxy terminal capping group of the peptide compound; and wherein the peptide compound upregulates expression of a gene encoding an antioxidative enzyme.

Preferably, compositions of the invention comprise an isolated peptide compound having an amino terminal capping group (designated R₁ in above formulas). More preferably, the amino terminal capping group is selected from the group consisting of a lipoic acid moiety (Lip, in reduced or oxidized form); a glucose-3-O-glycolic acid moiety (Gga); 1 to 6 naturally occurring L-amino acids (SEQ ID NO:6); an acyl group of the formula R₃—CO—, where CO is a carbonyl group, and R₃ is a hydrocarbon chain having from 1 to 25 carbon atoms, and preferably 1 to 22 carbon atoms, and where the hydrocarbon chain may be saturated or unsaturated and branched or unbranched; and combinations thereof. When the amino terminal capping group is an acyl group, preferably it is an acetyl or a fatty acyl group. Even more preferably, the amino terminal capping group is an acyl group selected from the group consisting of acetyl (acyl moiety of acetic acid), palmitoyl (acyl moiety of palmitic acid, Palm), and docosahexaenoyl (acyl moiety of docosahexaenoic acid, DHA). In yet another preferred embodiment, when the amino terminal capping group is 1 to 6 amino acids (SEQ ID NO:7), wherein the amino acids are selected from the group consisting of lysine, arginine, and a combination of lysine and arginine.

In another preferred embodiment, a composition of the invention comprises an isolated peptide compound that comprises a carboxy terminal capping group (designated R₂ in the above formulas) selected from the group consisting of a primary amine or a secondary amine.

Peptide compounds useful in the compositions and methods of the invention may be prepared and used as one or more various salt forms, including salts of alkali metals, salts of akaline metals, acetate salts, and trifluoroacetic acid salts, as suitable for a particular intended use of the peptide compound.

A peptide compound of the invention may also be linked to one or more other molecules that serve as detectable labels. Such labeled compounds may be particularly useful in analytical or preparative methods to track or detect the peptide compound during a procedure or protocol.

In addition, other compounds may be present in the compositions of the invention as deemed appropriate for a particular use. Such other components that may be present in compositions of the invention include, without limitation, salts, buffers, reducing agents, dietary supplements, other pharmaceutically active compounds, one or more other pharmaceutically acceptable excipients or carriers for various formulations, and combinations thereof.

The invention also provides methods of counteracting the effects of ROS and free radicals in cells and tissues comprising contacting the cells or tissues with an isolated peptide compound described herein. Compositions of the invention comprise an isolated peptide compound that stimulates (upregulates) expression of a gene(s) encoding superoxide dismutase (SOD) and/or catalase (CAT), which enzyme(s) plays a role in detoxifying ROS and free radicals in cells and tissues of animals, including humans and other mammals. Preferably, gene expression for both SOD and CAT proteins are upregulated by contacting cells or tissues with a composition comprising a peptide compound of this invention.

A preferred method of counteracting the effects of ROS and free radicals in cells and tissues comprises contacting cells or tissues with a composition comprising an isolated peptide compound of the invention to elevate the expression of a gene(s) encoding SOD and/or CAT to sufficiently high levels to provide increased detoxification of ROS and free radicals compared to cells or tissues not contacted with the composition.

Patients having a variety of diseases or conditions have been found to possess undesirable levels of ROS and/or free radicals. In a preferred embodiment of the invention, a composition comprising a peptide compound described herein may be used in a treatments for such diseases. In particular, a composition comprising a peptide compound described herein may be administered to an individual therapeutically to counteract the effects of ROS and free radicals present in the body and/or prophylactically to decrease or prevent an undesirable elevation in the levels of ROS and free radicals associated with particular diseases, conditions, drug treatments, or disorders. Specifically, this invention provides methods in which a composition comprising an isolated peptide compound described herein is administered to an individual to treat or prevent a disease or condition that is characterized by the generation of toxic levels of ROS and/or free radicals. Such diseases or conditions may be a condition related to aging, disease, or trauma, including but not limited to tissue degeneration during aging (senescence), cognitive degeneration during aging (senescence), senility, age-related motor impairment (e.g., ambulatory degeneration), Tardive dyskinesia, cerebral ischemia (stroke), myocardial infarct (heart attack), head trauma, brain and/or spinal cord trauma, reperfusion damage, oxygen toxicity in premature infants, Huntington's disease, Parkinson's disease, amyotrophic lateral sclerosis (ALS), Alzheimer's disease, diabetes, ulcerative colitis, human leukemia and other cancers characterized by elevation of ROS or free radicals, age-related elevation of ROS or free radicals, Down syndrome, macular degeneration, cataracts, schizophrenia, epilepsy, septic shock, polytraumatous shock, burn injuries and radiation-induced elevation of ROS and free radicals (including UV-induced skin damage).

In a particularly preferred embodiment, this invention provides methods in which a composition comprising an isolated peptide compound described herein is administered to an individual to lessen or eliminate side effects caused by drug regimens that generate ROS and/or free radicals. A number of drugs have been found to cause undesirable elevation of levels of ROS or free radicals as a toxic side effect. Such drugs include doxorubicin, daunorubicn, BCNU (cannustine) and related compounds such as methyl-BCNU and CCNU, and neuroleptics, such as clozapine. As an adjuvant to such therapies, isolated peptide compounds of this invention can be administered to an individual receiving such drug regimens to decrease, prevent, or eliminate the severity of such damaging side effects. Accordingly, the isolated peptide compounds of this invention may be administered to treat or prevent drug-induced elevation of ROS or free radicals, such as occurs during treatment with neuroleptic drugs as in Tardive dyskinesia.

In yet another embodiment, the isolated peptide compounds described herein are used as an alternative or adjuvant to nonsteroidal, anti-inflammatory drugs (NSAIDs) to treat pain from wounds, arthritis, and other inflammatory conditions in which ROS and free radicals play a role.

A preferred embodiment of the invention provides compositions comprising an isolated peptide compound of the invention in a pharmaceutically acceptable buffer which can be administered to an individual to eliminate, reduce, or prevent the generation of toxic levels of ROS or free radicals in cells or tissues, even when a specific disease has not yet been diagnosed or the full extent of an injury has not been fully understood.

Certain isolated peptide compounds present in the compositions of the invention may also have the ability to restore intracellular calcium ion to normal levels. Such peptide compounds are particularly useful to treat stroke.

### BRIEF DESCRIPTION OF THE DRAWINGS

FIG. 1 shows bar graphs depicting dose-dependent SOD expression in brain tissue extracts from 18-month old C57 Black mice that received PEP-1 (0.25 mg of PEP-1/kg body weight, oral gavage) on various administration schedules for a period 14 days. Extracts from brains of the mice were analyzed by Western immunoblot, and levels of SOD expression quantitated by densitometry measurements of the immunoblot. Group 1 animals were untreated (control); Group 2, PEP-1 administered once per week, Group 3, PEP-1 administered twice per week; Group 4, PEP-1 administered every other day; Group 5, PEP-1 administered daily. Data is presented as SOD band intensity for animals (n=3) of each treatment group relative to that for control Group 1 (untreated). “M” is SOD standard run alongside the samples in the immunoblot. Numbers above bars for M, Group 3, and Group 4 data provide selected numerical-fold increase in SOD level relative to Group 1. See text for details.

FIG. 2 shows bar graphs depicting dose-dependent SOD expression in brain tissue extracts from young (4 month old) and old (18 month old) C57 Black mice treated with various doses of PEP-1. Extracts from brains of the mice were analyzed by Western immunoblot, and levels of SOD expression quantitated by densitometry measurements of the immunoblot. Animals 1-3, young untreated mice; animals 4-6, old, untreated mice (control); animals 7-9, old mice, treated daily with PEP-1 (0.33 mg/kg); animals 10-12, old mice, treated daily with PEP-1 (3.3 mg/kg). Data is presented as SOD band intensity for each animal of each treatment group relative to that for old, untreated animals 4-6. “M” is SOD standard run alongside the samples in the immunoblot. “SOD-Related” is a breakdown fragment of SOD. See text for details.

FIG. 3 shows bar graphs depicting percent (%) survival of animals in groups (n=10) of young (4 month old) and old (16 month old) C57 Black mice that received various doses of PEP-1 by oral gavage for 70 days (2.5 months). Group 1 animals were untreated, young mice; Group 2 animals were untreated, old mice; Group 3 animals received a daily dose of PEP-1 of 0.025 mg/kg; Group 4 animals received a daily dose of PEP-1 of 0.075 mg/kg; Group 5 animals received a daily dose of PEP-1 of 0.25 mg/kg; Group 6 animals received a daily dose of PEP-1 of 0.75 mg/kg; and Group 7 animals received a daily dose of PEP-1 of 2.5 mg/kg. See text for details.

FIG. 4 shows bar graphs depicting locomotor activity score (Horizontal Count/Min) for C57 Black mice treated with various oral doses of PEP-1 as described for FIG. 3. Asterisk (*), P<0.05 compared with young control (student T-test following ANOVA). See text for details.

### DETAILED DESCRIPTION

This invention is based on the discovery of isolated peptide compounds that increase the expression of one or both genes encoding a complementary pair of enzymes, i.e., superoxide dismutase (SOD) and catalase (CAT), which are major components of the antioxidative defense mechanism or system in cells and tissues to detoxify reactive oxygen species (ROS) and free radicals. ROS and free radicals are generated during electron transport and normal respiration and other metabolic processes, including during the metabolism of various drugs, and must be rapidly detoxified to prevent permanent and continuing damage to cells and tissues. In addition, a number of diseases or conditions, including the aging process (senescence), are characterized by an elevation of ROS and/or free radicals to toxic levels that in fact damages cells or tissues and can lead to impairment of various functions. Accordingly, the peptide compounds described herein are valuable therapeutic and prophylactic compounds for counteracting the generation of harmful levels of ROS and free radicals in an individual.

In order that the invention may be better understood, the following terms are defined.

Abbreviations: Amino acid residues described herein may be abbreviated by the conventional three-letter or one-letter abbreviation know in the art (see, e.g., Lehninger, A. L., Biochemistry, second edition (Worth Publishers, Inc., New York, 1975), p. 72). Other abbreviations used herein include: “DHA” for the acyl form of docosahexaenoic acid moiety (i.e., docosahexaenoyl moiety); “Lip” for the acyl form of lipoic acid moiety, “Palm” for the acyl form of palmitic acid moiety (i.e., a palmitoyl group); “Ac” for the acyl form of acetic acid (i.e., an acetyl moiety); “Gga” for a glucose-3-O-glycolic acid moiety; “SOD” for super oxide dismutase (an antioxidative enzyme); “CAT” for catalase (an antioxidative enzyme); “GAPDH” for glyceraldehyde-3-phosphate dehydrogenase; and “ROS” for reactive oxygen species. Still other abbreviations are indicated as needed elsewhere in the text.

“Hydrocarbon” refers to either branched or unbranched and saturated or unsaturated hydrocarbon chains. Preferred hydrocarbon chains found in some of the peptide compounds described herein contain between 1 and 25 carbon atoms. More preferred are hydrocarbon chains between 1 and 22 carbon atoms.

“Reactive oxygen species” or “ROS”, as understood and used herein, refers to highly reactive oxygen compounds that are generated in the course of normal electron transport system during respiration, during aging, in a disease, or during treatment with certain therapeutic agents for a particular disorder. ROS include, but are not limited to, the superoxide anion (O₂.⁻), hydrogen peroxide (H₂O₂), singlet oxygen, lipid peroxides, and peroxynitrite. ROS are capable of causing oxidative damage to molecules, cells, and tissues.

“Free radical”, as understood and used herein, refers to any atom or any molecule or compound that possesses an odd (unpaired) electron. By this definition, the superoxide anion is also considered a negatively charged free radical. The free radicals of particular interest to this invention are highly reactive, highly oxidative molecules that are formed or generated during normal metabolism, during aging, in a diseased state, or during treatment with chemotherapeutic drugs. Such free radicals are highly reactive and capable of causing oxidative damage to molecules, cells and tissues. One of the most common and potentially destructive types of the free radicals other than the superoxide anion is a hydroxyl radical. Typically, the generation of ROS, such as superoxide anion or singlet oxygen, also leads to one or more other harmful free radicals as well. Accordingly, phrases such as “ROS and/or free radicals” or “ROS and other free radicals”, as understood and used herein, are meant to encompass any or all of the entire population of highly reactive, oxidative molecular species or compounds that may be generated in a particular metabolic state or condition of cells and tissues of interest (see, for example, Somani et al, “Response of Antioxidant System To Physical and Chemical Stress,” In Oxidants, Antioxidants, and Free Radicals, chapter 6: 125-141 (Taylor & Francis, Washington, D.C., 1997)).

“Oxygen radical scavengers” or “lazaroids” are a class of compounds that have an ability to scavenge and detoxify ROS and free radicals. Vitamins A, C, E, related antioxidant compounds, such as β-carotene and retinoids, and lipoic acid are also members of this large class of compounds, as are antioxidative enzymes, such as SOD and CAT. In healthy individuals, sufficient levels of antioxidative enzymes and other lazaroids are normally present both intracellularly and extracellularly to efficiently scavenge sufficient amounts of ROS and free radicals to significant oxidative damage to cells and tissues.

“Peptide compound”, as understood and used herein, refers to any compound described herein that contains at least 4 amino acids linked by peptide bonds and that upregulates expression of a gene encoding an antioxidative enzyme. “Peptide compound” includes unmodified or underivatized peptides, typically containing fewer than about 20, and preferably fewer than 12 amino acids. Peptide compounds of the invention include “derivatives” or “derivatized peptide compounds” of the invention, which are peptide compounds that are modified to contain one or more-chemical moieties other than amino acids that are linked, preferably covalently, to a peptide at an amino terminal amino acid residue, a carboxy terminal amino acid residue, or at an internal amino acid residue of the peptide. Such modifications include, without limitation, conservative amino acid substitutions, addition of a protective or capping group on a reactive moiety in the peptide, addition of a detectable label, and other changes that do not adversely destroy the activity of the peptide compound (i.e., its ability to upregulate expression of a gene encoding an antioxidative enzyme, such as SOD and/or CAT, to enhance the antioxidative activity in cells or tissues).

An “amino terminal capping group” of a peptide compound described herein is any chemical compound or moiety that is covalently linked or conjugated to the amino terminal amino acid residue of a peptide compound. An amino terminal capping group may be useful to inhibit or prevent intramolecular cyclization or intermolecular polymerization, to promote transport of the peptide compound across the blood-brain barrier (BBB), to protect the amino terminus from an undesirable reaction with other molecules, to provide additional antioxidative activity, or to provide a combination of these properties. A peptide compound of this invention that possesses an amino terminal capping group may possess other beneficial activities as compared with the uncapped peptide, such as enhanced efficacy or reduced side effects. For example, amino terminal capping groups used in the peptide compounds described herein may also possess antioxidative activity in their free state (e.g., lipoic acid, “Lip”, is a known scavenger of ROS and free radicals) and thus, may improve or enhance the antioxidative activity provided by the peptide compound in its uncapped form. Examples of amino terminal capping groups that are useful in preparing peptide compounds and compositions according to this invention include, but are not limited to, 1 to 6 naturally occurring L-amino acid residues (SEQ ID NO:6), preferably, 1-6 lysine residues (SEQ ID NO:7), 1-6 arginine residues (SEQ ID NO:7), or a combination of lysine and arginine residues (SEQ ID NO:7); urethanes; urea compounds; lipoic acid (“Lip”); glucose-3-O-glycolic acid moiety (“Gga”); or an acyl group that is covalently linked to the amino terminal amino acid residue of a peptide, wherein such acyl groups useful in the compositions of the invention may have a carbonyl group and a hydrocarbon chain that ranges from one carbon atom (e.g., as in an acetyl moiety) to up to 25 carbons (e.g., palmitoyl group, “Palm” (16:0) and docosahexaenoyl group, “DHA” (C22:6-3)). Furthermore, the carbon chain of the acyl group may be saturated, as in Palm, or unsaturated, as in DHA. It is understood that when an acid, such as docosahexaenoic acid, palmitic acid, or lipoic acid is designated as an amino terminal capping group, the resultant peptide compound is the condensed product of the uncapped peptide and the acid.

A “carboxy terminal capping group” of a peptide compound described herein is any chemical compound or moiety that is covalently linked or conjugated to the carboxy terminal amino acid residue of the peptide compound. The primary purpose of such a carboxy terminal capping group is to inhibit or prevent intramolecular cyclization or intermolecular polymerization, to promote transport of the peptide compound across the blood-brain barrier, and to provide a combination of these properties. A peptide compound of this invention possessing a carboxy terminal capping group may also possess other beneficial activities as compared with the uncapped peptide, such as enhanced efficacy, reduced side effects, enhanced hydrophilicity, enhanced hydrophobicity, or enhanced antioxidative activity (e.g., if the carboxy terminal capping moiety possesses its own source of reducing potential, such as one or more sulfhydryl groups). Carboxy terminal capping groups that are particularly useful in the peptide compounds described herein include primary or secondary amines that are linked by an amide bond to the α-carboxyl group of the carboxy terminal amino acid of the peptide compound. Other carboxy terminal capping groups useful in the invention include aliphatic primary and secondary alcohols and aromatic phenolic derivatives, including flavenoids, with 1 to 26 carbon atoms, which form esters when linked to the carboxylic acid group of the carboxy terminal amino acid residue of a peptide compound described herein.

“Effective amount” means an amount of a compound necessary to produce a desired effect. An effective amount of a peptide compound of the invention is the amount of the peptide compound that must be administered to cells, tissues, or an individual to produce an upregulation of the expression of a one or more genes encoding an antioxidative enzyme, e.g., SOD and/or CAT.

“Isolated peptide compound”, as used and understood herein, means a peptide compound comprising a peptide as described herein and which peptide is not present in a natural state, e.g., an isolated peptide compound is not present as part of a larger, naturally-occurring molecule, as a natural component of a biological source (e.g., cell, tissue, virus), or in an unfractionated extract from a biological source. Thus, an isolated peptide compound of the invention may be a purified, non-naturally occurring fragment of a naturally occurring protein or may be completely synthetic, i.e., having an amino acid sequence that is not found in nature and only produced using a peptide synthesis procedure.

“Pharmaceutical”, “pharmaceutically active compound”, and “pharmaceutical drug”, are synonymous and refer to any composition or compound that may be employed to treat a disease or condition in humans and/or other animals (i.e., in veterinary medicine). Such commonly known groups of pharmaceutically active compounds include, without limitation, psychotropic compounds (e.g., mood altering drugs), anti-cancer compounds, antibiotics, anti-ulcer drugs, anti-viral drugs, immunostimulatory compounds, immunosuppressive compounds, and anti-atherogenic compounds.

“Radiation”, as understood and used herein, means any type of propagating or emitted energy wave or energized particle, including electromagnetic radiation, ultraviolet radiation (UV), and other sunlight-induced radiation and radioactive radiation. The effects of such radiation may affect the surface or underlayers of the skin or may produce systemic damage at a remote site in the body.

“Upregulate” and “upregulation”, as understood and used herein, refer generally to an elevation in the level of expression of a gene in a cell or tissue. An elevation of gene expression is correlated and detected herein by higher levels of expression of the gene's product, e.g., a transcript or a protein, so that the terms “upregulate” and “upregulation” may be properly applied to describe an elevation in the level of expression of a gene's product as well. Peptide compounds described herein are capable of upregulating expression of a gene(s) encoding the antioxidative enzyme superoxide dismutase (SOD) and/or catalase (CAT) beyond the levels normally found in cells or tissues that have not been treated (contacted) with the peptide compounds. Thus, an elevation in the level of SOD or CAT mRNA transcript; in SOD or CAT gene product (protein) synthesis; or in the level of SOD or CAT enzyme activity indicates an upregulation of expression of a gene(s) encoding an antioxidative enzyme. Expression of SOD and CAT genes can be detected by a variety of methods including, but not limited to, Northern blotting to detect MRNA transcripts encoding SOD and/or CAT, Western immunoblotting to detect the gene product, i.e., SOD and/or CAT protein, or standard assays for SOD or CAT enzymatic activities.

Other terms will be evident as used in the following description.

Peptide Compounds and Compositions

The invention provides isolated peptide compounds described herein for use in compositions and/or methods that upregulate SOD and/or CAT gene expression in eukaryotic cells. Upregulating levels of SOD and/or CAT in cells or tissues provides an enhanced detoxification system to prevent, reduce, or eliminate the harmful oxidative activity of ROS and free radicals. Preferred peptides and peptide compounds of this invention upregulate both SOD and CAT.

The invention provides compositions comprising an isolated peptide compound having the formula:
R₁ Asp Gly Xaa₃ Xaa₄ Xaa₅ R₂ (SEQ ID NO:1),

wherein R₁ is absent or is an amino terminal capping group; Xaa₃ is Glu or Leu; Xaa₄ is Ala or Glu; Xaa₅ is absent, Leu, or Ala; and R₂ is absent or is a carboxy terminal capping group of the peptide compound; and wherein the peptide compound upregulates expression of a gene encoding an antioxidative enzyme. Preferably, such compositions comprise an isolated peptide compound of any of the following formulas:

| | |
|---|---|
| R₁ Asp Gly Glu Ala R₂, | (SEQ ID NO: 2) |
| R₁ Asp Gly Glu Ala Leu R₂, | (SEQ ID NO: 3) |
| R₁ Asp Gly Leu Glu Ala R₂, | (SEQ ID NO: 4) |

wherein R₁ is absent or is an amino terminal capping group of the peptide compound and R₂ is absent or is a carboxy terminal capping group of the peptide compound; and wherein the peptide compound upregulates expression of a gene encoding an antioxidative enzyme.

More generally, the invention provides compositions that comprise an isolated peptide compound having any of the following amino acid “core sequences”:

| | |
|---|---|
| Asp Gly Glu Ala, | (SEQ ID NO: 2) |
| Asp Gly Glu Ala Leu, | (SEQ ID NO: 3) |
| Asp Gly Leu Glu Ala, | (SEQ ID NO: 4) |

wherein the peptide compound upregulates expression of a gene encoding an antioxidative enzyme. Peptides comprising or consisting essentially of a core sequence may be used in a variety of compositions and methods for increasing antioxidative enzyme activity in cells or tissues to counteract the toxic effects of ROS and free radicals. It is understood that peptide compounds of the invention used in the compositions and methods of the invention may contain one or more additional amino acids linked at the amino terminal and/or carboxy terminal amino acids of a “core sequence” of amino acids (such as amino acid sequences of SEQ ID NOS:2, 3, and 4, above), provided the peptide compound still upregulates expression of a gene encoding an antioxidative enzyme to increase antioxidative enzyme activity in a cell or tissue. For example, a preferred embodiment of the invention provides compositions that comprise an isolated peptide compound having the formula:
R₁ Xaa₁ Xaa₂ Asp Gly Xaa₅ Xaa₆ Xaa₇ Xaa₈ Xaa₉ Xaa₁₀ Xaa₁₁ R₂ (SEQ ID NO:5),
wherein R₁ is absent or is an amino terminal capping group; Xaa₁ is absent or any amino acid; Xaa₂ is absent or any amino acid; Xaa₅ is Glu or Leu; Xaa₆ is Ala or Glu; Xaa₇ is absent, Leu, or Ala; Xaa₈ is absent or is any amino acid; Xaa₉ is absent or is any amino acid; Xaa₁₀ is absent or is any amino acid; Xaa₁₁ is absent or is any amino acid; and R₂ is absent or is a carboxy terminal capping group of the peptide compound; and wherein the peptide compound upregulates expression of a gene encoding an antioxidative enzyme.

The isolated peptide compounds described herein are preferably less than about 20, and, in order of increasing preference, less than about 12, 11, 10, 9, 8, 7, or 6 amino acids in length and are able to upregulate expression of a gene for SOD and/or CAT in cells or tissues.

Antioxidative activity in cells or tissues may be measured by standard methods in vitro, e.g., in tissue culture or by analysis of tissue or cell sample obtained from an individual. Methods to detect increased antioxidative activity include, but are not limited to, SOD or CAT enzyme activity assays; immunoblots for SOD and/or CAT production (e.g., Western blots, ELISA); and Northern blots to detect or measure levels of mRNA transcripts for the SOD and/or CAT genes.

Particularly preferred peptide compounds of the invention show upregulation activity at low concentrations, i.e., in the range of nanograms (ng) of peptide compound per milliliter (ml) or less. Such high potency is similar to that exhibited by various hormones, such as luteinizing hormone releasing hormone (LHRH) or human growth hormone. Accordingly, the peptide compounds described herein may be prepared, stored, and used employing much of the available technology already applied to the preparation, storage, and administration of known therapeutic hormone peptides.

The peptide compounds described herein may contain a peptide to which additional modifications have been made, such as addition of chemical moieties at the amino terminal and/or carboxy terminal amino acid residues of the peptide, conservative amino acid substitutions or modifications of side chains of internal amino acid residues of the peptide that do not destroy the desired activity of the peptide. It has been observed that intramolecular cyclization and some intermolecular polymerizations of peptides tend to inactivate or decrease the activity of the peptides, so that the peptide will not effectively upregulate SOD or CAT. Accordingly, the most useful peptide compounds are the least susceptible to cyclization reactions or an undesired polymerization or conjugation with other peptide compound molecules. In addition to maintaining or enhancing the ability of peptide compounds described herein to upregulate expression of SOD and/or CAT, some modifications may advantageously confer additional benefits. For example, amino terminal capping groups may promote transport of the peptide compound across the blood-brain barrier (BBB) (see, e.g., PCT publication WO 99/26620). This property is particularly important when a peptide compound is used to upregulate SOD and CAT in brain tissue and parts of the central nervous system. Amino terminal capping groups that promote transport across the blood-brain barrier (BBB) may also prevent cyclization of the peptide compound to which they are attached or may prevent polymerization with other peptide compounds.

Preferred amino terminal capping groups include a lipoic acid moiety, which can be attached by an amide linkage to the α-amino group of the amino terminal amino acid of a peptide. Lipoic acid (“Lip”) in its free form possesses independent antioxidative activity and may enhance the antioxidative activity of the peptides of this invention when used as an amino terminal capping group. An amino terminally linked lipoic acid moiety may be in its reduced form where it contains two sulfhydryl groups or in its oxidized form in which the sulfhydryl groups are oxidized and form an intramolecular disulfide bond and, thereby, a heterocyclic ring structure. Another amino terminal capping group useful in preparing peptide compounds of the invention is a glucose-3-O-glycolic acid moiety (“Gga”), which can be attached in an amide linkage to the α-amino group of the amino terminal amino acid of a peptide compound. The glucose moiety may also contain further modifications, such as an alkoxy group replacing one or more of the hydroxyl groups on the glucose moiety.

Another example of an amino terminal capping group useful in the peptide compounds described herein is an acyl group, which can be attached in an amide linkage to the α-amino group of the amino terminal amino acid residue of a peptide compound. The acyl group has a carbonyl group linked to a saturated or unsaturated (mono- or polyunsaturated), branched or unbranched, hydrocarbon chain of 1 to 25 carbon atoms in length, and more preferably, the hydrocarbon chain of the acyl group is 1 to 22 carbon atoms in length, as in DHA. The acyl group preferably is acetyl or a fatty acid. The fatty acid used as the acyl amino terminal capping group may contain a hydrocarbon chain that is saturated or unsaturated and that is either branched or unbranched. Preferably the hydrocarbon chain is 1 to 25 carbon atoms in length, and more preferably the length of the hydrocarbon chain is 1-22 carbon atoms in length. For example, fatty acids that are useful, in their corresponding acyl form, as amino terminal capping groups linked to the peptide compounds of this invention include, but are not limited to: caprylic acid (C8:0), capric acid (C10:0), lauric acid (C12:0), myristic acid (C 14:0), palmitic acid (“Palm”) (C16:0), palmitoleic acid (C16:1), C 16:2, stearic acid (C18:0), oleic acid (C18:1), vaccenic acid (C18:1-7), linoleic acid (C18:2-6), α-linolenic acid (C18:3-3), eleostearic acid (C18:3-5), β-linolenic acid (C18:3-6), C18:4-3, gondoic acid (C20:1), C20:2-6, dihomo-γ-linolenic acid (C20:3-6), C20:4-3, arachidonic acid (C20:4-6), eicosapentaenoic acid (C20:5-3), docosenoic acid (C22: 1), docosatetraenoic acid (C22:4-6), docosapentaenoic acid (C22:5-6), docosapentaenoic acid (C22:5-3), docosahexaenoic acid (“DHA”) (C22:6-3), and nervonic acid (C24:1-9). Particularly preferred fatty acids used as acyl amino terminal capping groups for the peptide compounds described herein are palmitic acid (Palm), docosahexaenoic acid (DHA). DHA and, other fatty acids that may promote transport of molecules to which they are linked across the blood-brain barrier (see, e.g., PCT publication WO 99/40112 and PCT publication WO 99/26620). Accordingly, such fatty acyl moieties are particularly preferred when a peptide compound described herein will be administered to counteract the oxidative effects of ROS and free radicals in brain tissue and/or other parts of the central nervous system.

In addition, in certain cases the amino terminal capping group may be a lysine residue or a polylysine peptide, preferably where the polylysine peptide consists of two, three, four, five or six lysine residues, which can prevent cyclization, crosslinking, or polymerization of the peptide compound. Longer polylysine peptides may also be used. Another amino terminal capping group that may be used in the peptide compounds described herein is an arginine residue or a polyarginine peptide, preferably where the polyarginine peptide consists of two, three, four, five, or six arginine residues, although longer polyarginine peptides may also be used. An amino terminal capping group of the peptide compounds described herein may also be a peptide containing both lysine and arginine, preferably where the lysine and arginine containing peptide is two, three, four, five or six residue combinations of the two amino acids in any order, although longer peptides that contain lysine and arginine may also be used. Lysine and arginine containing peptides used as amino terminal capping groups in the peptide compounds described herein may be conveniently incorporated into whatever process is used to synthesize the peptide compounds to yield the derivatized peptide compound containing the amino terminal capping group.

The peptide compounds useful in the compositions and methods of the invention may contain a catboxy terminal capping group. The primary purpose of this group is to prevent intramolecular cyclization or inactivating intermolecular crosslinking or polymerization. However, as noted above, a carboxy terminal capping group may provide additional benefits to the peptide compound, such as enhanced efficacy, reduced side effects, enhanced antioxidative activity, and/or other desirable biochemical properties. An example of such a useful carboxy terminal capping group is a primary or secondary amine in an amide linkage to the carboxy terminal amino acid residue. Such amines may be added to the α-carboxyl group of the carboxy terminal amino acid of the peptide using standard amidation chemistry.

Cyclization, crosslinking, or polymerization of a peptide compound described herein may abolish all or so much of the activity of the peptide compound so that it cannot be used in the therapeutic or prophylactic compositions and methods of the invention.

In addition, peptide compounds described herein may contain one or more D-amino acid residues in place of one or more L-amino acid residues provided that the incorporation of the one or more D-amino acids does not abolish all or so much of the activity of the peptide compound that it cannot be used in the compositions and methods of the invention. Incorporating D-amino acids in place of L-amino acids may advantageously provide additional stability to a peptide compound, especially in vivo.

The peptide compounds can be made using standard methods or obtained from a commercial source. Direct synthesis of the peptides of the peptide compounds of the invention may be accomplished using conventional techniques, including solid-phase peptide synthesis, solution-phase synthesis, etc. Peptides may also be synthesized using various recombinant nucleic acid technologies, however, given their relatively small size and the state of direct peptide synthesis technology, a direct synthesis is preferred and solid-phase synthesis is most preferred. In solid-phase synthesis, for example, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents, and reaction conditions used throughout the synthesis and are removable under conditions, which do not affect the final peptide product. Stepwise synthesis of the polypeptide is carried out by the removal of the N-protecting group from the initial (i.e., carboxy terminal) amino acid, and coupling thereto of the carboxyl end of the next amino acid in the sequence of the polypeptide. This amino acid is also suitably protected. The carboxyl group of the incoming amino acid can be activated to react with the N-terminus of the bound amino acid by formation into a reactive group such as formation into a carbodiimide, a symmetric acid anhydride, or an “active ester” group such as hydroxybenzotriazole or pentafluorophenyl esters. The preferred solid-phase peptide synthesis methods include the BOC method, which utilizes tert-butyloxycarbonyl as the α-amino protecting group, and the FMOC method, which utilizes 9-fluorenylmethloxycarbonyl to protect the α-amino of the amino acid residues, both methods of which are well-known by those of skill in the art (see, Stewart et al., Solid-Phase Peptide Synthesis (W. H. Freeman Co., San Francisco 1989); Merrifield, J. Am. Chem. Soc., 85:2149-2154 (1963); Bodanszky and Bodanszky, The Practice of Peptide Synthesis (Springer-Verlag, New York 1984), incorporated herein by reference). Amino terminal and carboxy terminal capping groups, if desired, may be added during or after peptide synthesis, depending on the specific moiety used as a capping group. For example, if the capping group is one or more amino acids, then such residues are simply incorporated into the protocol for synthesizing the peptide. If the capping group is not an amino acid, such as an acyl or amide group, it may be added after peptide synthesis using standard condensation or conjugation methods.

Peptide compounds according to the invention may also be prepared commercially by companies providing peptide synthesis as a service (e.g., BACHEM Bioscience, Inc., King of Prussia, Pa.; AnaSpec, Inc., San Jose, Calif.). Automated peptide synthesis machines, such as manufactured by Perkin-Elmer Applied Biosystems, also are available.

Peptide compounds useful in the compositions and methods of the invention may also be prepared and used in a salt form. Typically, a salt form of a peptide compound will exist by adjusting the pH of a composition comprising the peptide compound with an acid or base in the presence of one or more ions that serve as counter ion to the net ionic charge of the peptide compound at the particular pH. Various salt forms of the peptide compounds described herein may also be formed or interchanged by any of the various methods known in the art, e.g., by using various ion exchange chromatography methods. Cationic counter ions that may be used in the compositions described herein include, but are not limited to, amines, such as ammonium ion; metal ions, especially monovalent or divalent ions of alkali metals (e.g., sodium, potassium, lithium, cesium), alkaline earth metals (e.g., calcium, magnesium, barium), transition metals (e.g., iron, manganese, zinc, cadmium, molybdenum), other metals (e.g., aluminum); and combinations thereof Anionic counter ions that may be used in the compositions described herein include, but are not limited to, chloride, fluoride, acetate, trifluoroacetate, phosphate, sulfate, carbonate, citrate, ascorbate, sorbate, glutarate, ketoglutarate, and combinations thereof. Trifluoroacetate salts of peptide compounds described herein are typically formed during purification in trifluoroacetic acid buffers using high-performance liquid chromatography (HPLC). While generally not suited for in vivo use, trifluoroacetate salt forms of the peptide compounds described herein may be conveniently used in various in vitro cell culture studies or assays performed to test the activity or efficacy of a peptide compound of interest. The peptide compound may then be converted from the trifluoroacetate salt (e.g., by ion exchange methods) to or synthesized as a salt form that is acceptable for pharmaceutical or dietary supplement (nutraceutical) compositions.

After being produced or synthesized, a peptide compound that is useful in the compositions and methods of the invention may be purified using methods known in the art. Such purification should provide a peptide compound of the invention in a state dissociated from significant or detectable amounts of undesired side reaction products; unattached or unreacted moieties used to modify the peptide compound; and dissociated from other undesirable molecules, including but not limited to other peptides, proteins, nucleic acids, lipids, carbohydrates, and the like. Standard methods of peptide purification may be employed to obtained isolated peptide compounds of the invention, including but not limited to various high-pressure (or performance) liquid chromatography (HPLC) and non-HPLC peptide isolation protocols, such as size exclusion chromatography, ion exchange chromatography, phase separation methods, electrophoretic separations, precipitation methods, salting in/out methods, immunochromatography, and/or other methods.

A particularly preferred method of isolating peptide compounds useful in compositions and methods of the invention employs reversed-phase HPLC using an alkylated silica column such as C₄-, C₈- or C₁₈-silica. A gradient mobile phase of increasing organic content is generally used to achieve purification, for example, acetonitrile in an aqueous buffer, usually containing a small amount of trifluoroacetic acid. Ion-exchange chromatography can also be used to separate peptide compounds based on their charge. The degree of purity of the peptide compound may be determined by various methods, including identification of a major large peak on HPLC. A peptide compound that produces a single peak that is at least 95% of the input material on an HPLC column is preferred. Even more preferable is a polypeptide that produces a single peak that is at least 97%, at least 98%, at least 99% or even 99.5% of the input material on an HPLC column.

In order to ensure that a peptide compound obtained using any of the techniques described above is the desired peptide compound for use in compositions and methods of the present invention, analysis of the compound's composition determined by any of a variety of analytical methods known in the art. Such composition analysis may be conducted using high resolution mass spectrometry to determine the molecular weight of the peptide. Alternatively, the amino acid content of a peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to determine definitely the sequence of the peptide. Since some of the peptide compounds contain amino and/or carboxy terminal capping groups, it may be necessary to remove the capping group or the capped amino acid residue prior to a sequence analysis. Thin-layer chromatographic methods may also be used to authenticate one or more constituent groups or residues of a desired peptide compound. Purity of a peptide compound may also be assessed by electrophoresing the peptide compound in a polyacrylamide gel followed by staining to detect protein components separated in the gel.

The various peptide compounds described herein are useful in the compositions and methods of the invention to upregulate the expression of a gene(s) encoding SOD and/or CAT and thereby generate antioxidative activity to counteract the undesirable and destructive oxidative activity of ROS and free radicals, e.g., as generated in the aging process (senescence), disease, and various drug treatments.

Preferred peptide compounds, excluding any amino and/or carboxy terminal capping group (i.e., the “core sequence”), are less than about 20 amino acids in length, and more preferably, less than 12 amino acids in length. Even more preferred, such core sequences are less than 11, 10, 9, 8, 7, or even 6 amino acids in length. Most preferably, an isolated peptide compound useful in the compositions and methods of the invention has a core sequence of 4 or 5 amino acids in length, such as an amino acid core sequence selected from the group consisting of Asp Gly Glu Ala (SEQ ID NO:2), Asp Gly Glu Ala Leu (SEQ ID NO:3), and Asp Gly Leu Glu Ala (SEQ ID NO:4). Any amino terminal and/or carboxy terminal capping group described herein may be added to such preferred peptide compounds, provided the capping group does not destroy the ability to upregulate expression of SOD and/or CAT enzymes in a cell or tissue and provided the capping group does not also react with other groups of the peptide to produce a significant or toxic amount of undesirable cyclization or polymerization.

Biological and Biochemical Activities

The peptide compounds useful in the compositions and methods of the invention have the ability to upregulate SOD and/or CAT in cells and/or tissues, especially mammalian cells, provided the cells contain at least one functional gene encoding a SOD and/or CAT enzyme protein. A functional gene is one, which not only encodes the particular enzyme, but also provides the necessary genetic information within and without the coding sequence so that transcription of the gene can occur and so that the mRNA transcript can be translated into a functioning gene product e.g., in this case, an antioxidative enzyme.

Certain preferred peptide compounds described herein are able to upregulate expression of both SOD and CAT, when functional genes for the enzymes are present in the cells of interest. Advantageously, upregulation of SOD and CAT together provide particularly enhanced efficacy in detoxifying undesired ROS and free radicals. Without wishing to be bound by theory, when the level of SOD protein increases as a result of SOD upregulation, it is believed that superior antioxidative efficacy is achieved when there is also an increase in CAT levels. Upregulation of a gene for CAT increases the capacity to neutralize and detoxify the additional hydrogen peroxide and other ROS or free radicals that can be generated by enhanced SOD activity. The peptide compounds described herein having both SOD and CAT upregulation activity provide cells and tissues with a full complement of enhanced antioxidative enzyme activity to detoxify ROS and free radicals. For example, contacting mammalian cells in tissue culture with a peptide compound described herein having both SOD and CAT upregulation activity typically results in at least about a 2-fold and, in increasing order of-preference, at least about a 3-fold, 4-fold, and 6 to 8-fold increase in the levels of SOD and CAT mRNA transcripts and about a 2-fold and, in increasing order of preference, at least about a 3-fold, 4-fold, 6-fold, 8-fold, 10-fold and 12- to 14-fold increase in the levels of SOD and CAT protein, as detected by immunoblotting and compared to untreated cells. Such increase in SOD and CAT gene expression levels provides a cell with a significantly enhanced capability for detoxifying ROS and free radicals without adverse effects.

Expression of genes encoding SOD and CAT can be measured by a variety of methods. Standard enzymatic assays are available to detect levels of SOD and CAT in cell or tissue extracts or biological fluids (Fridovich, Adv. Enzymol., 41:35-97 (1974); Beyer & Fridovich, Anal. Biochem., 161:559-566 (1987)). In addition, antibodies to SOD and CAT are available or readily made. Using such antibodies specific for each protein, standard immunoblots (e.g., Western blots) and other immunological techniques can be used to measure levels of SOD and CAT in various mixtures, cell extracts, or other sample of biological material. Provided there is no evidence of a defect in the translation machinery of the cells of interest, the levels of expression of genes encoding SOD and CAT can also be measured by detecting levels of mRNA transcripts using standard Northern blot or standard polymerase chain reaction (PCR) methods for measuring specific MRNA species (e.g., RT-PCR).

Therapeutic and Prophylactic Applications

The isolated peptide compounds of the invention upregulate SOD and/or CAT in cells and tissues of animals, such as humans and other mammals. Preferably, the isolated peptide compounds of this invention upregulate both SOD and CAT. As noted above, SOD and CAT comprise components of the body's major enzymatic antioxidative activities that are able to detoxify ROS and free radicals by reducing such molecules to less reactive and less harmful compounds. The contribution of ROS and other free radicals to the progression of various disease states and side effects of drugs is now well known.

For example, elevated levels of ROS and free radicals are generated in cells and tissues during reperfusion after an ischemic event. Such increased levels of ROS and free radicals can cause considerable damage to an already stressed or debilitated organ or tissue. The peptide compounds of this invention, which upregulate SOD and/or CAT, may be used to treat reperfusion injuries that occur in diseases and conditions such as stroke, heart attack, renal disease, and kidney transplants. If the ischemic event has already occurred as in stroke and heart attack, a composition comprising a peptide compound described herein may be administered to the individual to detoxify the elevated ROS and free radicals already present or emerging in the blood and affected tissues or organs. Alternatively, if the ischemic event is anticipated as in organ transplantation, then a composition comprising a peptide compound described herein may be administered prophylactically prior to the operation or ischemic event.

Although a major application is in the treatment of ischemia-reperfusion injury, the peptide compounds described herein may be used to treat any disease or condition associated with undesirable levels of ROS and free radicals or to prevent any disease, disorder or condition caused by undesirable levels of ROS and free radicals. According to the invention, the peptide compounds described herein may also be administered to provide a therapeutic or prophylactic treatment of elevated ROS and other free radicals associated with a variety of other diseases and conditions, including, but not limited to, oxygen toxicity in premature infants, burns and physical trauma to tissues and organs, septic shock, polytraumatous shock, head trauma, brain trauma, spinal cord injuries, Parkinson's disease, amyotrophic lateral sclerosis (ALS), Alzheimer's disease, age-related elevation of ROS and free radicals, senility, ulcerative colitis, human leukemia and other cancers, Down syndrome, arthritis, macular degeneration, schizophrenia, epilepsy, radiation damage (including UV-induced skin damage), and drug-induced increase in ROS and free radicals.

A progressive rise of oxidative stress due to the formation of ROS and free radicals occurs during aging (see, e.g., Mecocci, P. et al., Free Radic. Biol. Med., 28: 1243-1248 (2000)). This has been detected by finding an increase in the formation of lipid peroxidates in rat tissues (Erdincler, D. S., et al., Clin. Chim. Acta, 265: 77-84 (1997)) and blood cells in elderly human patients (Congi, F., et al., Presse. Med., 24: 1115-1118 (1995)). A recent review (Niki, E., Intern. Med., 39: 324-326 (2000)) reported that increased tissue damage by ROS and free radicals could be attributed to decreased levels of the antioxidative enzymes SOD and CAT that occurs during aging. For example, transgenic animals, generated by inserting extra SOD genes into the genome of mice were found to have decreased levels of ROS and free radical damage. Such animals also had an extended life span. More recent evidence indicated that administration of a small manganese porphyrin compound, which mimics SOD activity, led to a 44% extension of life span of the nematode worm Caenorhabditis elegans (S. Melow, et al., Science, 289: 1567-1569 (2000)). As noted above, an increasing number of studies have also shown a correlation between oxidative tissue damage and age-associated brain dysfunction, as evidenced by age-related loss of various cognitive and motor functions, and/or a progressive increase in oxidatively modified DNA and proteins during aging (see, e.g., Coyle et al., Science, 262: 689-695 (1993); Halliwell, J. Neurochem., 59: 1609-1623 (1992); Sohal et al., Free Radical Biol. Med., 10: 495-500 (1990); Sohal et al., Mech. Ageing Dev., 76: 215-224 (1994); Ames, Free Radical Res. Commun., 7: 121-128 (1989); and Stadtman, Science, 257: 1220-1224 (1992)). Accordingly, the peptide compounds described herein, which are able to upregulate expression of SOD and/or CAT genes to produce increased levels of antioxidative enzymes, are also well suited for use in methods of preventing and/or counteracting increased tissue damage, loss of cognitive function, loss of motor function, and/or decreased life expectancy due to elevated levels of ROS and free radicals that accompany the aging process.

A variety of drugs in current therapeutic use produce tissue-specific toxic side effects that are correlated with an elevation in the levels of ROS and other free radicals. Such drugs include neuroleptics, antibiotics, analgesics, and other classes of drugs. The tissues affected by such drug-induced toxicities can include one or more of the major organs and tissues, such as brain, heart, lungs, liver, kidney, and blood.

One of the most dangerous side effects of a drug has been reported for the neuroleptic, clozapine, which was the first drug with major potential as an anti-schizophrenic therapeutic activity (see, Somani et al., In Oxidants, Antioxidants And Free Radicals (S. I. Baskin And H. Salem, eds.) (Taylor And Francis, Washington D.C., 1997), pages 125-136). Approximately 1-2% of clozapine-treated patients develop agranulocytosis, which is correlated with the production of ROS (Fischer et. al., Molecular Pharm., 40:846-853, 1991). According to the invention, a peptide compound as described herein is administered to clozapine-treated patients to upregulate the SOD and/or CAT, which counteracts the undesirable and harmful increase in ROS and other free radicals and, thereby, reduces the risk of developing agranulocytosis.

Another side effect of schizophrenic patients receiving neuroleptics is Tardive dyskinesia, which is a debilitating disease manifested by various uncontrollable oral, facial, and/or trunk movements. Many patients, especially veterans in hospital, suffer permanent disability from this unfortunate, drug-induced disease. Previous studies on Tardive dyskinesia were focused on the loss of dopamine neurons (see, for example, Morganstem and Glazer, Arch. Gen. Psychiatr., 50: 723-733 (1993)). However, more recent studies have demonstrated that the primary defect in brains of such patients is the overproduction of the excitotoxic amino acid glutamate in the presynaptic input to the striatal dopaminergic neurons. Notably, this overproduction of glutamate produces excitotoxic effects on dopamine cells by causing a high increase in ROS and free radicals (see, Tsai et al., Am. J. Psychiatr., 155: 1207-1253 (1998)). Accordingly, the peptide compounds of this invention may be administered to patients receiving neuroleptics to upregulate SOD and/or CAT and thereby provide the enhanced antioxidative activities to counteract the oxidative effects of the elevated levels of ROS and free radicals.

According to the methods of the invention, peptide compounds described herein may be administered to an individual before, contemporaneously with, or after administration of a therapeutic drug whose use has been correlated with the undesirable side effect of elevation in the levels of ROS and other free radicals. Such drugs include, but are not limited to those listed in Table 1 (see, Somani et al., 1997), which also lists any known manifested toxicity or side effect.

TABLE 1

| | ROS or Reactive Free Radical | |
|---|---|---|
| DRUG | or Toxic Result | Toxicity |
| clozapine | ROS and free radicals | agranulocytosis |
| doxorubin | superoxide anion, | cardiac |
| (anthrcyclines) | hydroxyl radical | |
| bleomycin | superoxide anion | pulmonary |
| mytomycin | free radical | |
| cisplatin | probably free radical | nephrotoxicity, |
| | | otototoxicity |
| BCNU (carmustine) | methyl radical | neurotoxicity |
| procarbazine | free radical | neurotoxicity |
| acetaminophen | reactive intermediate metabolites | hepatic |
| | of drug | |
| isoniazid | free radical | hepatic |
| ethanol | α-hydroxy ethyl radical | hepatic, |
| | | neurotoxicity |
| physostigmine | eseroline to catechol to quinones | neurotoxicity |
| quinones | reactive metabolites of drug | neurotoxicity |
| morphine | covalent binding reactions | neurotoxicity |
| nitrofurantoin | oxidant | pulmonary |
| paraquat | oxidant | pulmonary |
| parathion | reactive metabolites of drug | neruotoxicity |
| carbon tetrachloride | trichloromethyl radical | hepatic |
| (CCl₄) | | |
| polycyclic aromatic | reactive epoxides | hepatic |
| hydrocarbons | | |
| nitrofurazone | ROS and free radicals | pulmonary |
| metronidazole | ROS and free radicals | pulmonary |
| 6-hydroxydopamine | ROS and free radicals | neurotoxin |
| 4-hydroxyanisole | free radicals | |
| etoposide (VP-16) | hydroxyl radicals | |
| benzidine | free radicals | bladder |
| | | carcinogen |
| aminopyrine | free radicals | agranulocytosis |
| clozaril | free radicals | agranulocytosis |
| phenylhydrazine | ROS and free radicals | hemolytic anemia |
| 3-methylindole | free radicals | pulmonary |
| probucol | free radicals | |
| ferrous sulfate | hydroxyl radicals | iron overload |
| methimazole | free radicals | |
| chloroprazine | free radicals | phototoxicity, |
| | | photoallergy |
| salicylanilides | free radicals | photoallergy |
| mitoxantrone | free radicals | |
| daunomycin | ROS and free radicals | cardiotoxicity |

Pharmaceutical Applications

Pharmaceutical compositions of this invention comprise any of the isolated peptide compounds of the present invention, or pharmaceutically acceptable salts thereof, as the “active ingredient” of the pharmaceutical compositions. Pharmaceutical compositions of the invention may further comprise one or more other pharmaceutically acceptable ingredient, such as an excipient (a compound having a desirable property, but other than the active ingredient), carrier, adjuvant, or vehicle.

Pharmaceutical compositions of this invention can be administered to mammals, including humans, in a manner similar to other therapeutic, prophylactic, or diagnostic agents, and especially therapeutic hormone peptides. The dosage to be administered, and the mode of administration will depend on a variety of factors including age, weight, sex, condition of the patient, and genetic factors, and will ultimately be decided by the attending physician or veterinarian. In general, dosage required for diagnostic sensitivity or therapeutic efficacy will likely range from about 0.001 to 25.0 mg/kg of host body mass.

Pharmaceutically acceptable salts of the peptide compounds of this invention include, for example, those derived from pharmaceutically acceptable inorganic and organic acids and bases. Examples of suitable acids include hydrochloric, hydrobromic, sulfuric, nitric, perchloric, fumaric, maleic, malic, pamoic, phosphoric, glycolic, lactic, salicylic, succinic, toluene-p-sulfonic, tartaric, acetic, citric, methanesulfonic, formic, benzoic, malonic, naphthalene-2-sulfonic, tannic, carboxymethyl cellulose, polylactic, polyglycolic, and benzenesulfonic acids. Other acids, such as oxalic, while not in themselves pharmaceutically acceptable, may be employed in the preparation of salts useful as intermediates in obtaining the compounds of the invention and their pharmaceutically acceptable acid addition salts. Salts derived from appropriate bases include alkali metal (e.g., sodium), alkaline earth metal (e.g., magnesium), ammonium and N—(C₁₋₄ alkyl)₄⁺ salts.

This invention also envisions the “quatemization” of any basic nitrogen-containing groups of a peptide compound disclosed herein, provided such quatemization does not destroy the ability of the peptide compound to upregulate expression of genes encoding SOD and CAT. Such quatemization may be especially desirable where the goal is to use a peptide compound containing only positively charged residues. As noted above, in a most preferred embodiment of the invention, when charged amino acid residues are present in a peptide compound described herein, they are either all basic (positively charged) or all acidic (negatively) which prevents formation of cyclic peptide compounds during storage or use. Typically, cyclic forms of the peptide compounds are inactive and potentially toxic. Thus, a quaternized peptide compound is a preferred form of a peptide compound containing basic amino acids. Even more preferred is the quaternized peptide compound in which the carboxy terminal carboxyl groupd is converted to an amide to prevent the carboxyl group from reacting with any free amino groups to form a cyclic compound. Any basic nitrogen can be quaternized with any agent known to those of ordinary skill in the art including, for example, lower alkyl halides, such as methyl, ethyl, propyl and butyl chloride, bromides and iodides; dialkyl sulfates including dimethyl, diethyl, dibutyl and diamyl sulfates; long chain halides such as decyl, lauryl, myristyl and stearyl chlorides, bromides and iodides; and aralkyl halides including benzyl and phenethyl bromides. Water or oil-soluble or dispersible products may be obtained by such quaternization or acids such as acetic acid and hydrochloric acid.

It should be understood that the peptide compounds of this invention may be modified by appropriate functionalities to enhance selective biological properties, and in particular the ability to upregulate SOD and/or CAT. Such modifications are known in the art and include those, which increase the ability of the peptide compound to penetrate or being transported into a given biological system (e.g., brain, central nervous system, blood, lymphatic system), increase oral availability, increase solubility to allow administration by injection, alter metabolism of the peptide compound, and alter the rate of excretion of the peptide compound. In addition, the peptide compounds may be altered to a pro-drug form such that the desired peptide compound is created in the body of the patient as the result of the action of metabolic or other biochemical processes on the pro drug. Such pro-drug forms typically demonstrate little or no activity in in vitro assays. Some examples of pro-drug forms may include ketal, acetal, oxime, and hydrazone forms of compounds which contain ketone or aldehyde groups. Other examples of pro-drug forms include the hemi-ketal, hemi-acetal, acyloxy ketal, acyloxy acetal, ketal, and acetal forms.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical compositions of this invention may be administered by a variety of routes or modes. These include, but are not limited to, parenteral, oral, intratracheal, sublingual, pulmonary, topical, rectal, nasal, buccal, sublingual, vaginal, or via an implanted reservoir. Implanted reservoirs may function by mechanical, osmotic, or other means. The term “parenteral”, as understood and used herein, includes intravenous, intracranial, intraperitoneal, paravertebral, periarticular, periostal, subcutaneous, intracutaneous, intra-arterial, intramuscular, intra-articular, intrasynovial, intrasternal, intrathecal, and intralesional injection or infusion techniques. Such compositions are preferably formulated for parenteral administration, and most preferably for intravenous, intracranial, or intra-arterial administation. Generally, and particularly when administration is intravenous or intra-arterial, pharmaceutical compositions may be given as a bolus, as two or more doses separated in time, or as a constant or non-linear flow infusion.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant such as those described in Pharmacoplia Halselica.

The pharmaceutical compositions of this invention may be orally administered in any orally acceptable dosage form including, but not limited to, capsules, tablets, caplets, pills, aqueous or oleaginous suspensions and solutions, syrups, or elixirs. In the case of tablets for oral use, carriers, which are commonly used include lactose and corn starch. Lubricating agents, such as magnesium stearate, are also typically added. For oral administration in a capsule form, useful diluents include lactose and dried cornstarch. Capsules, tablets, pills, and caplets may be formulated for delayed or sustained release.

When aqueous suspensions are to be administered orally, the peptide compound is advantageously combined with emulsifying and/or suspending agents. If desired, certain sweetening and/or flavoring and/or coloring agents may be added. Formulations for oral administration may contain 10%-95% active ingredient, preferably 25%-70%. Preferably, a pharmaceutical composition for oral administration provides a peptide compound of the invention in a mixture that prevents or inhibits hydrolysis of the peptide compound by the digestive system, but allows absorption into the blood stream.

The pharmaceutical compositions of this invention may also be administered in the form of suppositories for vaginal or rectal administration. These compositions can be prepared by mixing a compound of this invention with a suitable non-irritating excipient, which is solid at room temperature but liquid at body temperature and therefore will melt in relevant body space to release the active components. Such materials include, but are not limited to, cocoa butter, beeswax and polyethylene glycols. Formulations for administration by suppository may contain 0.5%-10% active ingredient, preferably 1%-2%.

Topical administration of the pharmaceutical compositions of this invention may be useful when the desired treatment involves areas or organs accessible by topical application, such as in wounds or during surgery. For application topically, the pharmaceutical composition may be formulated with a suitable ointment containing the active components suspended or dissolved in a carrier. Carriers for topical administration of the peptide compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the peptide compounds suspended or dissolved in a pharmaceutically suitable carrier. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical composition may be formulated for topical or other application as a jelly, gel, or emollient, where appropriate. The pharmaceutical compositions of this invention may also be topically applied to the lower intestinal tract by rectal suppository formulation or in a suitable enema formulation. Topical administration may also be accomplished via transdermal patches. This may be useful for maintaining a healthy skin tissue and restoring oxidative skin damage (e.g., UV- or radiation-induced skin damage).

The pharmaceutical compositions of this invention may be administered nasally, in which case absorption may occur via the mucus membranes of the nose, or inhalation into the lungs. Such modes of administration typically require that the composition be provided in the form of a powder, solution, or liquid suspension, which is then mixed with a gas (e.g., air, oxygen, nitrogen, etc., or combinations thereof) so as to generate an aerosol or suspension of droplets or particles. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

Pharmaceutical compositions of the invention may be packaged in a variety of ways appropriate to the dosage form and mode of administration. These include but are not limited to vials, bottles, cans, packets, ampoules, cartons, flexible containers, inhalers, and nebulizers. Such compositions may be packaged for single or multiple administrations from the same container. Kits, of one or more doses, may be provided containing both the composition in dry powder or lyophilized form, as well an appropriate diluent, which are to be combined shortly before administration. The pharmaceutical composition may also be packaged in single use pre-filled syringes, or in cartridges for auto-injectors and needleless jet injectors.

Multi-use packaging may require the addition of antimicrobial agents such as phenol, benzyl alcohol, meta-cresol, methyl paraben, propyl paraben, benzalconium chloride, and benzethonium chloride, at concentrations that will prevent the growth of bacteria, fungi, and the like, but be non-toxic when administered to a patient.

Consistent with good manufacturing practices, which are in current use in the pharmaceutical industry and which are well known to the skilled practioner, all components contacting or comprising the pharmaceutical agent must be sterile and periodically tested for sterility in accordance with industry norms. Methods for sterilization include ultrafiltration, autoclaving, dry and wet heating, exposure to gases such as ethylene oxide, exposure to liquids, such as oxidizing agents, including sodium hypochlorite (bleach), exposure to high energy electromagnetic radiation, such as ultraviolet light, x-rays or gamma rays, and exposure to ionizing radiation. Choice of method of sterilization will be made by the skilled practioner with the goal of effecting the most efficient sterilization that does not significantly alter a desired biological function, i.e., the ability to upregulate SOD or CAT of the pharmaceutical agent in question. Ultrafiltration is a preferred method of sterilization for pharmaceutical compositions that are aqueous solutions or suspensions.

Details concerning dosages, dosage forms, modes of administration, composition and the like are further discussed in a standard pharmaceutical text, such as Remington's Pharmaceutical Sciences, 18th ed., Alfonso R. Gennaro, ed. (Mack Publishing Co., Easton, Pa. 1990), which is hereby incorporated by reference.

As is well known in the art, structure and biological function of peptides are sensitive to chemical and physical environmental conditions such as temperature, pH, oxidizing and reducing agents, freezing, shaking and shear stress. Due to this inherent susceptibility to degradation, it is necessary to ensure that the biological activity of a peptide compound used as a pharmaceutical agent be preserved during the time that the agent is manufactured, packaged, distributed, stored, prepared and administered by a competent practitioner. Many technical approaches have been developed to stabilize pharmaceutical proteins and peptides so as to preserve their biological potency and efficacy, and such stabilizing techniques may be applied to peptide compounds of the compositions and methods of the invention, including:
a) Freeze-drying and lyophilization (refer to Carpenter et al., Pharm. Res., 14(8): 969 (1997), incorporated by reference);
b) Addition of “stabilizers” to the aqueous solution or suspension of the peptide or protein. For example, U.S. Pat. No. 5,096,885 discloses addition of glycine, mannitol, pH buffers, and the non-ionic surfactant polysorbate 80 to human growth hormone as means to stabilize the protein during the process of filtration, vial filling, and cold storage or lyophilization; U.S. Pat. No. 4,297,344 discloses stabilization of coagulation factors II and VIII, antithrombin m and plasminogen against heat by adding selected amino acids and a carbohydrate; U.S. Pat. No. 4,783,441 discloses a method for prevention of denaturation of proteins such as insulin in aqueous solution at interfaces by the addition of surface acting substances, within a particular pH range; and U.S. Pat. No. 4,812,557 discloses a method of stabilizing interleukin-2 using human serum albumin;
c) Freeze/thaw methods wherein the peptide compound is mixed with a cryoprotectant and stored frozen at very low temperatures (e.g., −70° C.);
d) Cold, non-frozen storage (e.g., less than 4° C.), optionally with a cryoprotectant additive such as glycerol;
e) Storage in a vitrified, amorphous state, e.g., as described in U.S. Pat. No. 5,098,893;
f) Storage in a crystalline state; and
g) Incorporation into liposomes or other micelles.

Additional aspects of the invention will be further understood and illustrated in the following examples. The specific parameters included in the following examples are intended to illustrate the practice of the invention and its various features, and they are not presented to in any way limit the scope of the invention.

### EXAMPLES

### Example 1

### Synthesis of Representative Peptide Compounds

The following representative peptide compounds were synthesized by solid phase Merrifield synthesis (Merrifield, J. Am. Chem. Soc., 85:2149-2154 (1963)):

| | | |
|---|---|---|
| PEP-1: | [Ac] Asp Gly Glu Ala | (SEQ ID NO: 2) |
| PEP-2: | [Ac] Asp Gly Glu Ala Leu | (SEQ ID NO: 3) |
| PEP-3: | [Ac] Asp Gly Leu Glu Ala | (SEQ ID NO: 4) |

The amino terminal capping group “[Ac]” represents an acetyl moiety attached by acylation to the α-amino group of the amino terminal amino acid residue of the indicated peptide compounds (Shashoua and Hesse, Life Sci., 58: 1347-1357 (1996)).

The peptides were synthesized using standard procedures. Briefly, the peptides were synthesized using the solid phase Merrifield process (Merrifield, R. B., J. Am. Chem. Soc., 85:2149-2154 (1963)). This method allows the synthesis of a peptide of a specific amino acid sequence bound on a polymeric resin. Each newly synthesized peptide was then released from the resin by treating with trifluoroacetic acid (TFA). The resultant trifluoroacetic acid peptide salt was purified by ether precipitation according to standard procedures (see, E. Groos and Meienhofer, In The peptides, analysis, synthesis, biology, vol. 2, (Academic Press, New York 1983)).

For N-terminal substituted peptides (i.e., peptides containing an acyl amino terminal capping group), each peptide was synthesized with blocked side chains using solid phase Merrifield synthesis (see above). The bound peptide was then treated with an equimolar amount of an anhydride of one of the following acids: acetic acid in the presence of 4-dimethylamino pyridine under argon atmosphere. The reaction was carried out for about three hours to obtain N-terminal coupling. Evidence of complete N-terminal coupling was obtained prior to peptide isolation. This was established by monitoring the ninhydrin staining properties of the resin bound peptides using standard procedures (E. Kaiser, et al., Anal. Biochem., 34: 595-598 (1970)). The N-terminal coupled (capped) peptide molecule was then released from the resin by treatment with TFA and purified by precipitation with cold ether followed by HPLC using methanolic HCl (50:50) as the eluant. The final peptide products were white solids after lyophilization. Structures were confirmed by amino acid analyses, by migration as a single peak on HPLC, and molecular weight determinations by mass spectrometry. For most uses, it was essential to completely remove TFA from the peptide compound. This was achieved by repeated dissolution of the peptide in glacial acetic acid followed by concentration in vacuo in rotary evaporator. Complete absence of TFA was established by mass spectrometry.

### Example 2

### Upregulation of Superoxide Dismutase (SOD) and Catalase (CAT) in Mammalian Cells by Peptide Compound PEP-2 ([Ac] Asp Gly Glu Ala Leu (SEQ ID NO:3))

Primary cortical cultures were obtained by growing newborn rat brain cortical cells in Delbecco's modified Eagle medium supplemented with 100 units/ml of penicillin G, 100 μg/ml of streptomycin, and 10% fetal calf serum. The cells were isolated from the E-21 cortex of rat brain, plated at a density of 1×10⁵ per ml and grown to confluence within four to five days in an atmosphere containing air and 5% CO₂ at 37° C. as described in Cornell-Bell et al., Science, 247: 470473 (1990) and Cell Calcium, 12: 185-204 (1991). Cultures were grown in 20 ml flasks as a monolayer and then exposed to various concentrations of peptide for studies of the effects of PEP-2 on upregulation of the gene for SOD. Cultures of the rat brain cortical cells were incubated with 1 ng/ml, 10 ng/ml, and 100 ng/ml of peptide compound PEP-2 for durations of 5 hours. Control cultures were treated in the same manner, but were not incubated with PEP-2.

Cytoplasmic proteins were isolated according to published methods (Adams et al., J. Leukoc. Biol. 62: 865-873 (1997)). The cell cultures were washed once in phosphate buffer saline (PBS) containing 20 mM EDTA and then suspended in 250 μl of freshly prepared lysis buffer (20 mM Hepes, pH 7.9, 10 mM KCl, 300 mM NaCl, 1 mM MgCl₂, 0.1% Triton X-100 nonionic detergent, 20% glycerol, 0.5 mM dithiothreitol (DTT), freshly supplemented with inhibitors as described in Adams et al., J. Biol. Chem., 77: 221-233 (2000)). The suspensions were then incubated for at least 10 minutes on ice to lyse cells and then centrifuged (14,000×g for 5 minutes at 4° C.) to pellet cell debris. The supernatant cytoplasmic fractions were removed and stored as aliquots at −80° C. for analysis. The protein concentrations of the cytoplasmic fraction varied within 2-6 μg/μl.

The cytoplasmic proteins were separated by SDS-PAGE using 5 μg/lane on the gels for analysis by Western immunoblots. The gels were processed for Western immunoblots basically as described by Adams et al. (General Cellular Biochemistry, 77: 221-233 (2000)) to measure upregulation of SOD. SOD expression was detected in the Western blots using anti-SOD rabbit polyclonal antibody (Rockland, Inc., Gilbertville, Pa.). The Western blots were also analyzed by laser densitometry to quantify SOD protein upregulation. The control in these experiments was an identical culture flask, which was treated only with vehicle (i.e., buffer, no peptide compound). The results are shown in Table 2 below.

TABLE 2
PEP-2-Dependent Upregulation of SOD-1 in Rat Cortical Cells

| Dose | Fold-Increase in SOD Expression |
|---|---|
| 1 ng/ml | 1.8 |
| 10 ng/ml | 1.9 |
| 100 ng/ml | 2.1 |

Western blots showed antibody binding to a band migrating at 34 kDa (the molecular weight of SOD-1), and two lower molecular weight bands corresponding to smaller components recognized by the anti-SOD-1 antibody in cells from cultures incubated in the presence and absence of PEP-2 (data not shown). However, as shown in Table 2, the study on the rat brain cortical cell cultures showed that the level of immunoreactive (i.e., anti-SOD antibody reactive) protein that was synthesized in the cytoplasm was increased by 2-fold even in cells treated with the lowest PEP-2 concentration (i.e., 1 ng/ml). Clearly, a peptide compound of the invention can upregulate SOD-1, in the rat cortical cells.

### Example 3

### In Vivo Pharmacological Activity of PEP-1 and PEP-3 Peptide Compounds

In vivo experiments were carried out in Sprague-Dawley rats (300-325 g) with solutions of peptide compounds PEP-1 and PEP-3. The animals were injected intravenously (iv) via the tail vein with a peptide compound. Each animal received one injection of peptide compound in normal saline at total dose equivalent of 1.2 or 2.4 mg peptide compound/kg body weight. The animals were sacrificed by decapitation at 6 hours post injection and dissected to isolate brain and heart organs, which were frozen at −70° C. for subsequent analysis by Western immunoblots.

Each tissue was thawed and homogenized in a Down's homogenizer using ten volumes of homogenizer buffer (see, Adams et al., General Cellular Biochemistry, 77: 221-233 (2000); buffer as described in Adams et al., J. Leukoc. Biol., 62: 865-875 (1967)) to obtain a crude cytoplasmic fraction. The tissue homogenates were centrifuged (14,000×g for 5 minutes at 4° C.) to yield the supernatant purified cytoplasmic protein fractions for Western immunoblot analysis as described in Adams et al. (J. Cell. Biochem., 77: 221-233 (2000)). A 10 μg sample of each protein fraction was then separated by SDS polyacrylamide gel electrophoresis (SDS-PAGE) and analyzed for SOD and CAT content by Western blot assay as above.

Controls for measurement of unstimulated levels of SOD were obtained from two vehicle-only (i.e., no peptide compound), injected rats that were sacrificed at 6 hours post injection. Both had essentially the same unstimulated level of SOD. Standard quantities of each cytoplasmic fraction (10 μg) were loaded on a lane of a gel for electrophoretic separation and Western immunoblot analysis (Adams et al., J. Cell. Biochem., 77: 221-233 (2000)). The stained gels were photographed and scanned by laser densitometry to quantify intensities in comparison to enzyme levels for control vehicle treated rats.

Table 3 and Table 4 show the upregulation data for SOD and CAT in rat brain and heart, respectively, compared to control animals that received only the injection vehicle without peptide compound.

TABLE 3
Effect of Peptide Compounds on SOD and CAT
Upregulation in Rat Brain

| Peptide | Amino Acid | Dose | SOD | CAT |
|---|---|---|---|---|
| Compound | Sequence | (mg/kg) | (Fold increase) | (Fold increase) |
| PEP-1 | [Ac]DGEA | 1.2 | 1.0 | 1.0 |
| | (SEQ ID NO:2) | | | |
| PEP-1 | [Ac]DGEA | 2.4 | 3.1 | 2.1 |
| | (SEQ ID NO:2) | | | |
| PEP-3 | [Ac]DGLEA | 1.2 | 1.0 | 1.0 |
| | (SEQ ID NO:4) | | | |
| PEP-3 | [Ac]DGLEA | 2.4 | 3.3 | 1.6 |
| | (SEQ ID NO:4) | | | |

TABLE 4
Effect of Peptide Compounds on SOD and CAT
Upregulation in Rat Heart

| Peptide | Amino Acid | Dose | SOD | CAT |
|---|---|---|---|---|
| Compound | Sequence | (mg/kg) | (Fold increase) | (Fold increase) |
| PEP-1 | [Ac]DGEA | 1.2 | 1.0 | 1.1 |
| | (SEQ ID NO:2) | | | |
| PEP-1 | [Ac]DGEA | 2.4 | 1.7 | 2.3 |
| | (SEQ ID NO:2) | | | |
| PEP-3 | [Ac]DGLEA | 1.2 | 1.2 | 1.0 |
| | (SEQ ID NO:4) | | | |
| PEP-3 | [Ac]DGLEA | 2.4 | 1.7 | 2.8 |
| | (SEQ ID NO:4) | | | |

The data in Tables 3 and 4 show that administration of the peptide compounds PEP-1 and PEP-3 at a dose equivalent to 2.4 mg/kg body weight resulted in an approximately three-fold upregulation of SOD production in brain and approximately a two-fold upregulation in SOD production in heart, relative to the control. At the same dose, CAT production was upregulated by approximately two-fold in brain and by approximately two to three-fold in heart.

The above results indicate that the peptide compounds PEP-1 and PEP-3 are active in vivo in upregulating SOD and CAT in cells and tissues of critical organs. These findings demonstrate the potential for using compounds such as PEP-1 and/or PEP-3 in compositions for treatments that promote the defense of critical organs in whole organisms against ROS and free radicals.

### Example 4

### In Vivo Dose-Response Effect of Peptide Compound PEP-1 on SOD Levels in “Old” C57 Black Mice

This study was designed to examine the in vivo dose-response effect of a representative peptide compound, PEP-1 (see, Example 1), on the level of SOD in “old” adult individuals.

Adult 18-month old C57 Black mice (“old mice”) were treated with 0.25 mg/kg of PEP-1 by oral gavage for a period of 14 days. The animals belonged to the following 5 treatment groups (n=3 per group):
Group 1: Control, untreated
Group 2: Treatment once per week
Group 3: Treatment twice per week
Group 4: Treatment every other day
Group 5: Daily treatment

At the end of 14 days, all animals were sacrificed and their brains were harvested and the SOD level was analyzed by Western immunoblot as described above. A SOD standard (“M”) was also run alongside the samples. The SOD levels were quantitated by densitometry measurements of the blot.

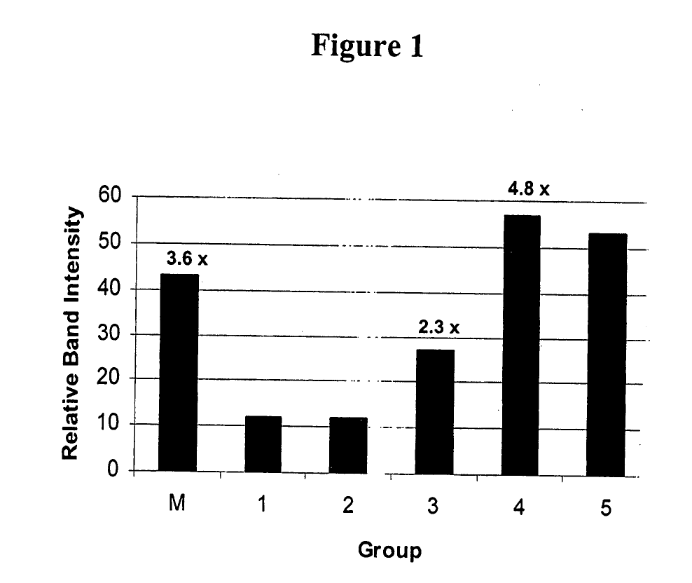

The results shown as bar graphs in FIG. 1 indicated that the SOD level was elevated over 4.5-fold in animals treated with PEP-1, either daily (Group 4) or every other day (Group 5) compared to control group animals (Group 1). In addition, a 2.3-fold increase in SOD level was noted in animals treated with the peptide twice per week (Group 3)(see, FIG. 1).

### Example 5

### In vivo Dose-Response Effect of Peptide Compound PEP-1 on SOD in “Old” Adult C57 Black Mice

As a further examination of the dose-response effect on upregulating SOD in “old” adult C57 Black mice, young and old animals were divided into the following treatment groups (n=3):
Animals 1-3: Young, 4-month old mice, untreated
Animals 4-6: Old, 18-month old mice, untreated
Animals 7-9: Old, 18-month old mice treated daily by oral gavage 0.33 mg/kg of PEP-1
Animals 10-1 2: Old, 18-month old mice treated daily by oral gavage 3.3 mg/kg of PEP-1

At the end of 30 days, all animals were sacrificed and their brains were harvested. SOD level was analyzed by Western immunoblot. A SOD standard (“M”) was also run alongside the samples. The SOD levels were quantitated by densitometry measurements of the immunoblot.

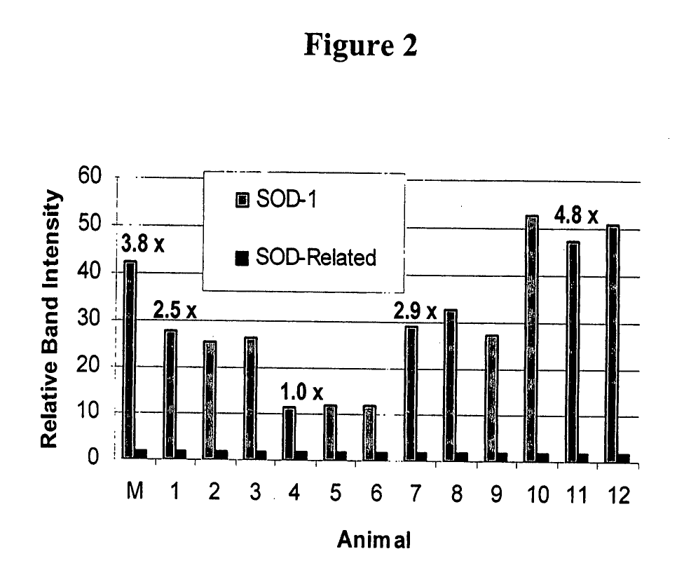

The results are shown in the bar graphs in FIG. 2. The brain tissue of young control (untreated) mice (animals 1-3) contained more than twice the level of SOD as is was found in old mice. A clear dose-response effect of PEP-1 on SOD expression was observed in the brain tissue of old mice in this study. The data in FIG. 2 show that the level of SOD expression in brain tissue of old mice that were treated daily with an oral dose of 3.3 mg/kg of PEP-1 (animals 10-12) was elevated 4.8-fold as compared to the untreated old control group animals (animals 4-6). In addition, a 2.9-fold increase in SOD level was noted in animals treated daily with an oral dose of 0.33 mg/kg of PEP-1. There was no change in SOD-related breakdown fragments as seen in the FIG. 2 (“SOD-Related”).

### Example 6

### In Vivo Effects of PEP-1 on Locomotor Activity and Longevity of Old C57 Black Mice

This study was designed to examine the in vivo effect of daily administration of a representative peptide compound, PEP-1, to reverse age-related declines in spontaneous behavioral arousal as measured by effect on locomotor activity and longevity of “old” adult individuals. In this study, C57 Black mice were divided into the following treatment groups (n=10 per group):
Group 1: Young, 4-month old mice, untreated
Group 2: Old, 16-month old mice, untreated
Group 3: Old, 16-month old mice treated daily by oral gavage with 0.025 mg/kg of PEP-1

Group 4: Old, 16-month old mice treated daily by oral gavage with 0.075 mg/kg of PEP-1
Group 5: Old, 16-month old mice treated daily by oral gavage with 0.25 mg/kg of PEP-1
Group 6: Old, 16-month old mice treated daily by oral gavage with 0.75 mg/kg of PEP-1
Group 7: Old, 16-month old mice treated daily by oral gavage with 2.5 mg/kg of PEP-1

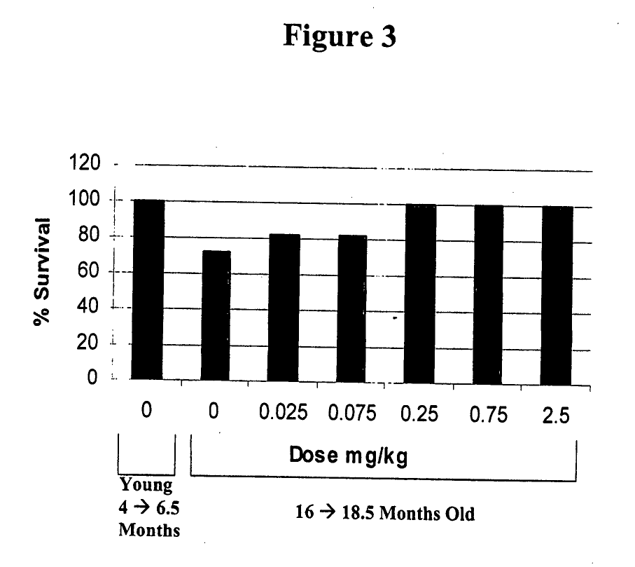

At the end of a 70-day treatment period, all of the young, untreated mice (Group 1) were alive (i.e., to an age of 6.5 months), but 3 of the 10 untreated old mice (Group 2) had died. Also, 2 animals in each of Groups 3 and 4 died during this same period. In Groups 5, 6 and 7, all of the old mice treated with PEP-1 survived the 70 days, i.e., to an old age of 18.5 months (see, FIG. 3).

Six weeks after assignment of the doses, surviving animals were examined in a standard locomotor activity test used to detect evidence of a reversal of age-related declines in spontaneous behavioral arousal (Forster et al., 1996; Dubey et al., Arch. Biochem. Biophys., 333: 189-197 (1996)). Arousal levels were measured using a Digiscan apparatus (Omnitech, model RXYZCM, Omnitech Electronics, Columbus, Ohio, USA). Each mouse was placed in a clear acrylic chamber (40.5 cm×40.5 cm×30.5 cm) for a 20-minute session. The acrylic chamber was placed in a metal frame lined with arrays of photocells. A wooden cabinet with sheets of insulation enclosed the acrylic chamber and the metal frame. Fans provided an 80-db ambient noise level within the chamber. Movements of the mouse within the horizontal plane interrupted the photobeams, which were automatically recorded by a computer within 1-minute time samples. The measure used in this study was the average horizontal photobeam interruptions per minute, over a 16-minute testing session (horizontal counts/min).

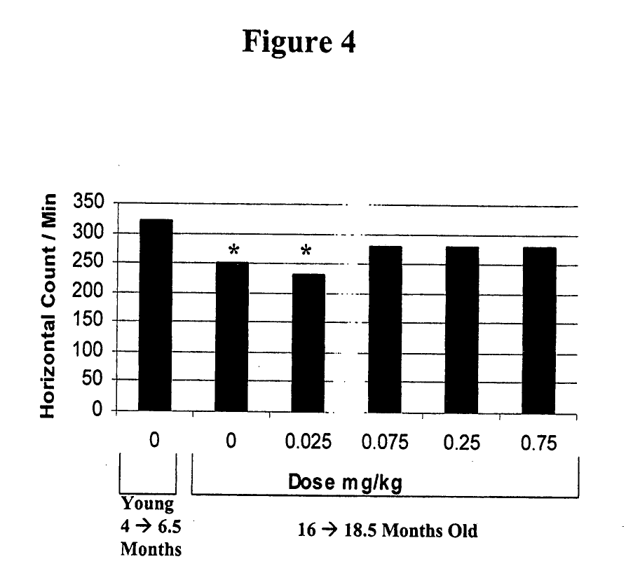

The locomotor activity test was performed on blinded Groups 1, 2, 3, 4, 5, and 6, (young untreated control, old untreated control, old treated with PEP-1 at 0.025 mg/kg 0.075 mg/kg, 0.25 mg/kg, and 0.75 mg/kg, respectively) six weeks after assignment of the doses. The results are shown in FIG. 4. A significant difference between the young and old untreated control groups was found (p<0.05), verifying previous findings of the age-associated decrease in spontaneous behavioral arousal. The 0.025 mg/kg treated animals (Group 3) failed to differ significantly from the old controls and were significantly different from the young controls. However, the horizontal activity of groups receiving from 0.075 to 0.75 mg/kg (Groups 4, 5, and 6) was higher than that of the old controls, and did not differ significantly from the young controls. These results indicated that there was a beneficial effect on behavioral arousal after six weeks of treatment with from 0.075 to 0.75 mg/kg of PEP-1.

The data from this study showed that treatment with PEP-1 increased the life span of old mice. Moreover, the data also indicated that treatment with PEP-1 significantly improved locomotor performance in old animals. Such results provide evidence for a beneficial, anti-aging effect of treatment with a peptide compound of the invention, such as PEP-1, which is able to upregulate expression of antioxidative enzymes in older individuals.

Other variations and embodiments of the invention described herein will now be apparent to those of ordinary skill in the art without departing from the scope of the invention or the spirit of the claims below.

All patents, applications, and publications cited in the above text are incorporated herein by reference.

1. A composition comprising an isolated peptide compound having the formula:
R₁ Asp Gly Xaa₃ Xaa₄ Xaa₅ R₂ (SEQ ID NO:1),
wherein R₁ is absent or is an amino terminal capping group; Xaa₃ is Glu or Leu; Xaa₄ is Ala or Glu; Xaa₅ is absent, Leu, or Ala; and R₂ is absent or is a carboxy terminal capping group; and wherein the peptide compound upregulates expression of a gene encoding an antioxidative enzyme.

2. A composition comprising an isolated peptide compound having a formula selected from the group consisting of:

| | |
|---|---|
| R₁ Asp Gly Glu Ala R₂, | (SEQ ID NO: 2) |
| R₁ Asp Gly Glu Ala Leu R₂, | (SEQ ID NO: 3) |
| and | |
| R₁ Asp Gly Leu Glu Ala R₂, | (SEQ ID NO: 4) |

wherein R₁ is absent or is an amino terminal capping group of the peptide compound and R₂ is absent or is a carboxy terminal capping group; and wherein the peptide compound upregulates expression of a gene encoding an antioxidative enzyme.

3. A composition comprising an isolated peptide compound having the formula:
R₁ Xaa₁ Xaa₂ Asp Gly Xaa₅ Xaa₆ Xaa₇ Xaa₈ Xaa₉ Xaa₁₀ Xaa₁₁ R₂ (SEQ ID NO:5),
wherein R₁ is absent or is an amino terminal capping group; Xaa₁ is absent or any amino acid; Xaa₂ is absent or any amino acid; Xaa₅ is Glu or Leu; Xaa₆ is Ala or Glu; Xaa₇ is absent, Leu, or Ala; Xaa₈ is absent or any amino acid; Xaa₉ is absent or any amino acid; Xaa₁₀ is absent or any amino acid; Xaa₁₁ is absent or any amino acid; and R₂ is absent or is a carboxy terminal capping group; and wherein the peptide compound upregulates expression of a gene encoding an antioxidative enzyme.

4. A composition comprising an isolated peptide compound consisting of an amino acid sequence selected from the group consisting of:

| | |
|---|---|
| Asp Gly Glu Ala, | (SEQ ID NO: 2) |
| Asp Gly Glu Ala Leu, | (SEQ ID NO: 3) |
| and | |
| Asp Gly Leu Glu Ala, | (SEQ ID NO: 4) |

wherein the peptide compound upregulates expression of a gene encoding an antioxidative enzyme.

5. The composition according to any one of claims 1, 2, 3, and 4, wherein the antioxidative enzyme is superoxide dismutase or catalase.

6. The composition according to any one of claims 1, 2, and 3, wherein R₁ is an amino terminal capping group selected from the group consisting of a reduced or oxidized lipoic acid moiety; a glucose-3-O-glycolic acid moiety; 1-6 lysine residues (SEQ ID NO:7); 1-6 arginine residues (SEQ ID NO:7); an acyl group R₃—CO—, where CO represents a carbonyl group and R₃ is a saturated or an unsaturated hydrocarbon chain having from 1 to 25 carbons, and combinations thereof.

7. The composition according to claim 6, wherein the amino terminal capping group is the R₃—CO acyl group wherein R₃ is a saturated or unsaturated hydrocarbon chain having 1 to 22 carbons.

8. The composition according to claim 6, wherein the amino terminal capping group R₁ is selected from the group consisting of acetyl, palmitoyl (Palm), and docosahexaenoyl (DHA).

9. The composition according to any one of claims 1, 2, and 3, wherein R₂ is the carboxy terminal capping group selected from the group consisting of a primary amine and a secondary amine.

10. A method of upregulating the level of expression of a superoxide dismutase gene, a catalase gene, or both, in cells or tissues comprising contacting cells or tissues with composition according to any one of claims 1-9 in an amount effective to upregulate expression of an antioxidative enzyme.

11. A method of counteracting the oxidative effects of reactive oxygen species and free radicals in mammalian cells or tissue comprising contacting the cells or tissue with a composition according to any one of claims 1-9 in an amount effective to upregulate expression of an antioxidative enzyme.

12. A method of reducing or preventing an undesirable elevation in the levels of reactive oxygen species and other free radicals in cells or tissues comprising contacting the cells or tissues with a composition according to any one of claims 1-9 in an amount effective to upregulate expression of an antioxidative enzyme.

13. A method of treating a disease or condition in a mammal exhibiting an undesirable elevation in levels of reactive oxygen species or other free radicals in cells or tissues of said mammal comprising administering to said mammal a composition according to any one of claims 1-9 in an amount effective to upregulate expression of an antioxidative enzyme.

14. The method according to claim 13, wherein said disease or condition is selected from the group consisting of cerebral ischemia, myocardial infarct, renal reperfusion, atherosclerosis, head trauma, brain trauma, spinal cord trauma, oxygen toxicity in premature infants, neurodegenerative disease, arthritis, inflammation, diabetes, ulcerative colitis, cancer, Down syndrome, macular degeneration, cataracts, schizophrenia, epilepsy, septic shock, polytraumatous shock, burn injuries, radiation-induced elevation of reactive oxygen species or other free radicals, and drug-induced elevation of reactive oxygen species or other free radicals.

15. The method according to claim 14, wherein said disease or condition is neurodegenerative disease.

16. The method according to claim 15, wherein said neurodegenerative disease is is selected from the group consisting of Huntington's disease, Parkinson's disease, and amyotrophic lateral sclerosis.

17. The method according to claim 13, wherein said disease or condition is a disease or condition related to the aging process.

18. The method according to claim 17, wherein said disease or condition related to the aging process is selected from the group consisting of decreased cognitive function, decreased motor function, senility, Alzheimer's disease, premature aging, and decreased life expectancy.

19. A method of treating pain in an individual comprising administering to the individual an effective amount of a peptide compound according to any of claims 1-9 in an amount effective to upregulate expression of an antioxidative enzyme.

20. A pharmaceutical composition comprising a peptide compound having the formula:
R₁ Xaa₁ Xaa₂ Asp Gly Xaa₅ Xaa₆ Xaa₇ Xaa₈ Xaa₉ Xaa₁₀ Xaa₁₁ R₂ (SEQ ID NO:5),
wherein R₁ is absent or is an amino terminal capping group; Xaa₁ is absent or any amino acid; Xaa₂ is absent or any amino acid; Xaa₅ is Glu or Leu; Xaa₆ is Ala or Glu; Xaa₇ is absent, Leu, or Ala; Xaa₈ is absent or any amino acid; Xaa₉ is absent or any amino acid; Xaa₁₀ is absent or any amino acid; Xaa₁₁ is absent or any amino acid; and R₂ is absent or is a carboxy terminal capping group.

21. The pharmaceutical composition of claim 20, wherein the said peptide compound having the formula selected from the group consisting of:

| | |
|---|---|
| R₁ Asp Gly Glu Ala R₂, | (SEQ ID NO: 2) |
| R₁ Asp Gly Glu Ala Leu R₂, | (SEQ ID NO: 3) |
| R₁ Asp Gly Leu Glu Ala R₂, | (SEQ ID NO: 4) |

wherein R₁ is absent or is an amino terminal capping group of the peptide compound and R₂ is absent or is a carboxy terminal capping group of the peptide compound.

22. The pharmaceutical composition of claim 20, wherein the said peptide compound upregulates expression of a gene encoding an antioxidative enzyme.

23. The pharmaceutical composition of claim 20, wherein the said peptide compound is capable of reducing or preventing reactive oxygen species.

24. The pharmaceutical composition of claim 20, wherein the said pharmaceutical composition is useful for treating or preventing a disease in a mammal.

25. A method for treating or preventing a disease in a mammal, comprising administrating to such a mammal an effective amount of the pharmaceutical composition of claim 20.

26. The method of claim 25, wherein the said mammal is human.

27. The method of claim 25, wherein the said disease is selected from the group consisting of cerebral ischemia, myocardial infarct, renal reperfusion, atherosclerosis, head trauma, brain trauma, spinal cord trauma, oxygen toxicity in premature infants, neurodegenerative disease, arthritis, inflammation, diabetes, ulcerative colitis, cancer, Down syndrome, macular degeneration, cataracts, schizophrenia, epilepsy, septic shock, polytraumatous shock, burn injuries, radiation-induced elevation of reactive oxygen species or other free radicals, and drug-induced elevation of reactive oxygen species or other free radicals.

28. The method of claim 25, wherein the said disease or condition is neurodegenerative disease.

29. The method of claim 28, wherein said neurodegenerative disease is selected from the group consisting of Huntington's disease, Parkinson's disease, and amyotrophic lateral sclerosis.

30. A pharmaceutical composition consisting essentially of a peptide compound having the formula:
R₁ Xaa₁ Xaa₂ Asp Gly Xaa₅ Xaa₆ Xaa₇ Xaa₈ Xaa₉ Xaa₁₀ Xaa₁₁ R₂ (SEQ ID NO:5),
wherein R₁ is absent or is an amino terminal capping group; Xaa₁ is absent or any amino acid; Xaa₂ is absent or any amino acid; Xaa₅ is Glu or Leu; Xaa₆ is Ala or Glu; Xaa₇ is absent, Leu, or Ala; Xaa₈ is absent or any amino acid; Xaa₉ is absent or any amino acid; Xaa₁₀ is absent or any amino acid; Xaa₁₁ is absent or any amino acid; and R₂ is absent or is a carboxy terminal capping group.

31. The pharmaceutical composition of claim 30, wherein the said peptide compound having the formula selected from the group consisting of:

| | |
|---|---|
| R₁ Asp Gly Glu Ala R₂, | (SEQ ID NO: 2) |
| R₁ Asp Gly Glu Ala Leu R₂, | (SEQ ID NO: 3) |
| R₁ Asp Gly Leu Glu Ala R₂, | (SEQ ID NO: 4) |

wherein R₁ is absent or is an amino terminal capping group of the peptide compound and R₂ is absent or is a carboxy terminal capping group of the peptide compound.

32. The pharmaceutical composition of claim 30, wherein the said peptide compound upregulates expression of a gene encoding an antioxidative enzyme.

33. The pharmaceutical composition of claim 30, wherein the said peptide compound is capable of reducing or preventing reactive oxygen species.

34. The pharmaceutical composition of claim 30, wherein the said pharmaceutical composition is useful for treating or preventing a disease in a mammal.

35. A method for treating or preventing a disease in a mammal, comprising administrating to such a mammal an effective amount of the pharmaceutical composition of claim 30.

36. The method of claim 35, wherein the said mammal is human.

37. The method of claim 35, wherein the said disease is selected from the group consisting of cerebral ischemia, myocardial infarct, renal reperfusion, atherosclerosis, head trauma, brain trauma, spinal cord trauma, oxygen toxicity in premature infants, neurodegenerative disease, arthritis, inflammation, diabetes, ulcerative colitis, cancer, Down syndrome, macular degeneration, cataracts, schizophrenia, epilepsy, septic shock, polytraumatous shock, burn injuries, radiation-induced elevation of reactive oxygen species or other free radicals, and drug-induced elevation of reactive oxygen species or other free radicals.

38. The method of claim 35, wherein the said disease or condition is neurodegenerative disease.

39. The method of claim 38, wherein said neurodegenerative disease is selected from the group consisting of Huntington's disease, Parkinson's disease, and amyotrophic lateral sclerosis.

40. An isolated pharmaceutical composition consisting essentially of a peptide compound having the formula:
R₁ Xaa₁ Xaa₂ Asp Gly Xaa₅ Xaa₆ Xaa₇ Xaa₈ Xaa₉ Xaa₁₀ Xaa₁₁ R₂ (SEQ ID NO:5),
wherein R₁ is absent or is an amino terminal capping group; Xaa₁ is absent or any amino acid; Xaa₂ is absent or any amino acid; Xaa₅ is Glu or Leu; Xaa₆ is Ala or Glu; Xaa₇ is absent, Leu, or Ala; Xaa₈ is absent or any amino acid; Xaa₉ is absent or any amino acid; Xaa₁₀ is absent or any amino acid; Xaa₁₁ is absent or any amino acid; and R₂ is absent or is a carboxy terminal capping group.

41. The pharmaceutical composition of claim 40, wherein the said peptide compound having the formula selected from the group consisting of:

| | |
|---|---|
| R₁ Asp Gly Glu Ala R₂, | (SEQ ID NO: 2) |
| R₁ Asp Gly Glu Ala Leu R₂, | (SEQ ID NO: 3) |
| R₁ Asp Gly Leu Glu Ala R₂, | (SEQ ID NO: 4) |

wherein R₁ is absent or is an amino terminal capping group of the peptide compound and R₂ is absent or is a carboxy terminal capping group of the peptide compound.

42. The pharmaceutical composition of claim 40, wherein the said peptide compound upregulates expression of a gene encoding an antioxidative enzyme.

43. The pharmaceutical composition of claim 40, wherein the said peptide compound is capable of reducing or preventing reactive oxygen species.

44. The pharmaceutical composition of claim 40, wherein the said pharmaceutical composition is useful for treating or preventing a disease in a mammal.

45. A method for treating or preventing a disease in a mammal, comprising administrating to such a mammal an effective amount of the pharmaceutical composition of claim 40.

46. The method of claim 45, wherein the said mammal is human.

47. The method of claim 45, wherein the said disease is selected from the group consisting of cerebral ischemia, myocardial infarct, renal reperfusion, atherosclerosis, head trauma, brain trauma, spinal cord trauma, oxygen toxicity in premature infants, neurodegenerative disease, arthritis, inflammation, diabetes, ulcerative colitis, cancer, Down syndrome, macular degeneration, cataracts, schizophrenia, epilepsy, septic shock, polytraumatous shock, burn injuries, radiation-induced elevation of reactive oxygen species or other free radicals, and drug-induced elevation of reactive oxygen species or other free radicals.

48. The method of claim 45, wherein the said disease or condition is neurodegenerative disease.

49. The method of claim 48, wherein said neurodegenerative disease is selected from the group consisting of Huntington's disease, Parkinson's disease, and amyotrophic lateral sclerosis.

50. An isolated composition comprising a peptide compound having the formula:
R₁ Xaa₁ Xaa₂ Asp Gly Xaa₅ Xaa₆ Xaa₇ Xaa₈ Xaa₉ Xaa₁₀ Xaa₁₁ R₂ (SEQ ID NO:5),
wherein R₁ is absent or is an amino terminal capping group; Xaa₁ is absent or any amino acid; Xaa₂ is absent or any amino acid; Xaa₅ is Glu or Leu; Xaa₆ is Ala or Glu; Xaa₇ is absent, Leu, or Ala; Xaa₈ is absent or any amino acid; Xaa₉ is absent or any amino acid; Xaa₁₀ is absent or any amino acid; Xaa₁₁ is absent or any amino acid; and R₂ is absent or is a carboxy terminal capping group.

51. The composition of claim 50, wherein the said peptide compound having the formula selected from a group consisting of:

| | |
|---|---|
| R₁ Asp Gly Glu Ala R₂, | (SEQ ID NO: 2) |
| R₁ Asp Gly Glu Ala Leu R₂, | (SEQ ID NO: 3) |
| R₁ Asp Gly Leu Glu Ala R₂, | (SEQ ID NO: 4) |

wherein R₁ is absent or is an amino terminal capping group of the peptide compound and R₂ is absent or is a carboxy terminal capping group of the peptide compound.

52. The composition of claim 50, wherein the said peptide compound upregulates expression of a gene encoding an antioxidative enzyme.

53. The composition of claims 50-52, wherein the composition is obtained from a natural source.

54. A method for preparing a pharmaceutical or dietary supplement composition, comprising mixing a peptide compound with a suitable vehicle, wherein the peptide compound having the formula:
R₁ Xaa₁ Xaa₂ Asp Gly Xaa₅ Xaa₆ Xaa₇ Xaa₈ Xaa₉ Xaa₁₀ Xaa₁₁ R₂ (SEQ ID NO:5),
wherein R₁ is absent or is an amino terminal capping group; Xaa₁ is absent or any amino acid; Xaa₂ is absent or any amino acid; Xaa₅ is Glu or Leu; Xaa₆ is Ala or Glu; Xaa₇ is absent, Leu, or Ala; Xaa₈ is absent or any amino acid; Xaa₉ is absent or any amino acid; Xaa₁₀ is absent or any amino acid; Xaa₁₁ is absent or any amino acid; and R₂ is absent or is a carboxy terminal capping group.

55. The method of claim 54, wherein the said peptide compound having the formula selected from a group consisting of:

| | |
|---|---|
| R₁ Asp Gly Glu Ala R₂, | (SEQ ID NO: 2) |
| R₁ Asp Gly Glu Ala Leu R₂, | (SEQ ID NO: 3) |
| R₁ Asp Gly Leu Glu Ala R₂, | (SEQ ID NO: 4) |

wherein R₁ is absent or is an amino terminal capping group of the peptide compound and R₂ is absent or is a carboxy terminal capping group of the peptide compound.

56. The method of claim 54, wherein the suitable vehicle is a pharmaceutically acceptable carrier or excipients.

57. The method of claim 54, wherein the suitable vehicle is derived from a natural source.

58. A dietary supplement composition comprising a peptide compound having the formula:
R₁ Xaa₁ Xaa₂ Asp Gly Xaa₅ Xaa₆ Xaa₇ Xaa₈ Xaa₉ Xaa₁₀ Xaa₁₁ R₂ (SEQ ID NO:5),
wherein R₁ is absent or is an amino terminal capping group; Xaa₈ is absent or any amino acid; Xaa₂ is absent or any amino acid; Xaa₅ is Glu or Leu; Xaa₆ is Ala or Glu; Xaa₇ is absent, Leu, or Ala; Xaa₈ is absent or any amino acid; Xaa₉ is absent or any amino acid; Xaa₁₀ is absent or any amino acid; Xaa₁₁ is absent or any amino acid; and R₂ is absent or is a carboxy terminal capping group.

59. The composition of claim 58, wherein the said peptide compound having the formula selected from a group consisting of:

| | |
|---|---|
| R₁ Asp Gly Glu Ala R₂, | (SEQ ID NO: 2) |
| R₁ Asp Gly Glu Ala Leu R₂, | (SEQ ID NO: 3) |
| R₁ Asp Gly Leu Glu Ala R₂, | (SEQ ID NO: 4) |

wherein R₁ is absent or is an amino terminal capping group of the peptide compound and R₂ is absent or is a carboxy terminal capping group of the peptide compound.

60. The composition of claim 58, wherein the said peptide compound upregulates expression of a gene encoding an antioxidative enzyme.

61. The composition of claim 58, wherein the said peptide compound is capable of reducing or preventing reactive oxygen species.

62. The composition of claim 58, wherein the said composition is useful for treating or preventing a disease in a mammal.

63. The composition of claim 58, wherein the said composition is obtained from a group consisting of plants and microorganisms.

64. The composition of claim 58, further comprising exogenously added peptide compound.


SEQUENCE LISTING (7 sequences)

SEQ ID NO: 1 - protein, 5 aa, Artificial Sequence
  upregulator of antioxidative enzyme expression
DGXXX

SEQ ID NO: 2 - protein, 4 aa, Artificial Sequence
  upregulator of antioxidative enzyme expression
DGEA

SEQ ID NO: 3 - protein, 5 aa, Artificial Sequence
  upregulator of antioxidative enzyme expression
DGEAL

SEQ ID NO: 4 - protein, 5 aa, Artificial Sequence
  upregulator of antioxidative enzyme expression
DGLEA

SEQ ID NO: 5 - protein, 11 aa, Artificial Sequence
  upregulator of antioxidative enzyme expression
XXDGXXXXXXX

SEQ ID NO: 6 - protein, 6 aa, Artificial Sequence
  amino terminal capping group
XXXXXX

SEQ ID NO: 7 - protein, 6 aa, Artificial Sequence
  amino terminal capping group
XXXXXX